(19) United States
(12) Patent Application Publication
(10) Pub. No.: US 2015/0088430 A1
(43) Pub. Date: Mar. 26, 2015

(54) METHODS FOR EVALUATING LUNG CANCER STATUS

(71) Applicant: ALLEGRO DIAGNOSTICS CORP, Maynard, MA (US)

(72) Inventor: Duncan H. Whitney, Sudbury, MA (US)

(21) Appl. No.: 14/397,431

(22) PCT Filed: Apr. 26, 2013
(86) PCT No.: PCT/US13/38449

§ 371 Date: Oct. 27, 2014

Related U.S. Application Data
(60) Provisional application No. 61/639,063, filed on Apr. 26, 2012.
(60) Provisional application No. 61/664,129, filed on Jun. 25, 2012.

(51) Int. Cl. C12Q 1/68; G06F 19/18; G06F 19/20; A61B 6/03; G06F 19/00

(52) U.S. Cl. CPC C12Q 1/6886; A61B 6/03; G06F 19/3431; G06F 19/20; G06F 19/18; C12Q 2600/118; C12Q 2600/158; C12Q 2600/16
USPC 702/19; 435/6.11; 506/9; 435/6.12; 536/24.31; 506/16

(57) ABSTRACT

The invention in some aspects provides methods of determining the likelihood that a subject has lung cancer based on the expression of informative-genes. In other aspects, the invention provides methods for determining an appropriate diagnostic intervention plan for a subject based on the expression of informative-genes. Related compositions and kits are provided in other aspects of the invention.

### RELATED APPLICATIONS

This application claims priority under 35 U.S.C. §119(e) of U.S. Provisional Patent Application No. 61/639,063, filed on Apr. 26, 2012 and entitled “METHODS FOR EVALUATING LUNG CANCER STATUS,” and U.S. Provisional Patent Application No. 61/664,129, filed on Jun. 25, 2012 and entitled “METHODS FOR EVALUATING LUNG CANCER STATUS.” Each of these applications is incorporated herein by reference in its entirety for all purposes.

### FIELD OF THE INVENTION

The invention generally relates to methods and compositions for assessing cancer risk using gene expression information.

### BACKGROUND OF INVENTION

A challenge in diagnosing lung cancer, particularly at an early stage where it can be most effectively treated, is gaining access to cells to diagnose disease. Early stage lung cancer is typically associated with small lesions, which may also appear in the peripheral regions of the lung airway, which are particularly difficult to reach by standard techniques such as bronchoscopy.

### SUMMARY OF INVENTION

Provided herein are methods for establishing appropriate diagnostic intervention plans and/or treatment plans for subjects, and for aiding healthcare providers in establishing appropriate diagnostic intervention plans and/or treatment plans. In some embodiments, the methods are based on an airway field of injury concept. In some embodiments, the methods involve establishing lung cancer risk scores based on expression levels of informative-genes. In some embodiments, the methods involve making a risk assessment based on expression levels of informative-genes in a biological sample obtained from a subject during a routine cell or tissue sampling procedure. In some embodiments, the biological sample comprises histologically normal cells. In some embodiments, aspects of the invention are based, at least in part, on a determination that expression levels of certain informative-genes in apparently histologically normal cells obtained from a first airway locus can be used to evaluate the likelihood of cancer at a second locus in the airway (for example, at a locus in the airway that is remote from the locus at which the histologically normal cells were sampled). In some embodiments, sampling of histologically normal cells (e.g., cells of the bronchus) is advantageous because tissues containing such cells are generally readily available, and thus it is possible to reproducibly obtain useful samples compared with procedures that involve obtaining tissues of suspicious lesions which may be much less reproducibly sampled. In some embodiments, the methods involve making a lung cancer risk assessment based on expression levels of informative-genes in cytologically normal appearing cells collected from the bronchi of a subject. In some embodiments, the informative-genes useful for predicting the risk of lung cancer are provided in Tables 4, 7-8, and 9-11.

In some embodiments, the informative-genes are selected from the group consisting of: BST1, APT12A, DEFB1, C3, TNFAIP2, SOD2, EPHX3, LST1, HCK, CA12, IRAK2, FMNL1, SERPING1, G0S2, and LCP2. In some embodiments, the informative-genes are selected from the group consisting of: TMTC2, SCHIP1, NMUR2, SORBS2, NPAS2, AKAP12, CSDA, SH3BGRL2, CD9, C9orf102, GRIK2, CAPN9, C19orf2, PRSS23, CA12, NCL, FUT8, PAWR, MTERFD3, RMND5A, OXR1, ALG1L, DAAM1, SLC26A2, AGPS, HDGFRP3, PLCB4, PAM, FOXJ3, TSPAN5, EDEM3, DEFB1, SLC17A5, ZBTB34, MYO1E, MIA3, and ZNF12. In some embodiments, the informative-genes are selected from the group consisting of: EPHX3, HLA-DQB2, BST1, ATP12A, HLA-DQB2, C3, CD82, INSR, PTPN7, FMNL1, IKBKE, RAC2, NINJ1, HLA-DPB1, MDK, ACSS2, HCK, GPRC5B, IRAK2, PLEK, COTL1, CYTH4, TNFAIP2, SCNN1B, LCP2, SOD2, HLA-DMB, CMTM1, SERPING1, CIITA, LILRA5, REC8, CORO1A, LST1, P2RY13, NCF4, G0S2, and TMC6. In some embodiments, the informative-genes are selected from the group consisting of: ACSS2, AKAP12, ATP12A, BST1, C3, CA12, CA8, CCDC81, CD82, EPHX3, ETS1, GPRC5B, HLA-DQB2, INSR, LOC339524, NKX3-1, NMUR2, SH3BGRL2, SLAMF7, and TSPAN5.

In some embodiments, appropriate diagnostic intervention plans are established based at least in part on the lung cancer risk scores. In some embodiments, the methods assist health care providers with making early and accurate diagnoses. In some embodiments, the methods assist health care providers with establishing appropriate therapeutic interventions early on in patient clinical evaluations. In some embodiments, the methods involve evaluating biological samples obtained during bronchoscopic procedures. In some embodiments, the methods are beneficial because they enable health care providers to make informative decisions regarding patient diagnosis and/or treatment from otherwise uninformative bronchoscopies. In some embodiments, the risk assessment leads to appropriate surveillance for monitoring low risk lesions. In some embodiments, the risk assessment leads to faster diagnosis, and thus, faster therapy for certain cancers.

Certain methods described herein, alone or in combination with other methods, provide useful information for health care providers to assist them in making diagnostic and therapeutic decisions for a patient. Certain methods disclosed herein are employed in instances where other methods have failed to provide useful information regarding the lung cancer status of a patient. Certain methods disclosed herein provide an alternative or complementary method for evaluating or diagnosing cell or tissue samples obtained during routine bronchoscopy procedures, and increase the likelihood that the procedures will result in useful information for managing a patient's care. The methods disclosed herein are highly sensitive, and produce information regarding the likelihood that a subject has lung cancer from cell or tissue samples (e.g., histologically normal tissue) that may be obtained from positions remote from malignant lung tissue. Certain methods described herein can be used to assess the likelihood that a subject has lung cancer by evaluating histologically normal cells or tissues obtained during a routine cell or tissue sampling procedure (e.g., standard ancillary bronchoscopic procedures such as brushing, biopsy, lavage, and needle-aspiration). However, it should be appreciated that any suitable tissue or cell sample can be used. Often the cells or tissues that are assessed by the methods appear histologically normal. In some embodiments, the subject has been identified as a candidate for bronchoscopy and/or as having a suspicious lesion in the respiratory tract.

In some embodiments, the methods disclosed herein are useful because they enable health care providers to determine appropriate diagnostic intervention and/or treatment plans by balancing the risk of a subject having lung cancer with the risks associated with certain invasive diagnostic procedures aimed at confirming the presence or absence of the lung cancer in the subject. In some embodiments, an objective is to align subjects with low probability of disease with interventions that may not be able to rule out cancer but are lower risk. In some embodiments, subjects with a relatively high probability of disease are subjected to more definitive interventions which are also significantly higher risk.

According to some aspects of the invention, methods are provided for evaluating the lung cancer status of a subject using gene expression information that involve one or more of the following acts: (a) obtaining a biological sample from the respiratory tract of a subject, wherein the subject has been referred for bronchoscopy (e.g., has been identified as having a suspicious lesion in the respiratory tract and therefore referred for bronchoscopy to evaluate the lesion), (b) subjecting the biological sample to a gene expression analysis, in which the gene expression analysis comprises determining the expression levels of a plurality of informative-genes in the biological sample, (c) computing a lung cancer risk score based on the expression levels of the plurality of informative-genes, (d) determining that the subject is in need of a first diagnostic intervention to evaluate lung cancer status, if the level of the lung cancer risk score is beyond (e.g., above) a first threshold level, and (e) determining that the subject is in need of a second diagnostic intervention to evaluate lung cancer status, if the level of the lung cancer risk score is beyond (e.g., below) a second threshold level. In some embodiments, the methods further comprise (f) determining that the subject is in need of a third diagnostic intervention to evaluate lung cancer status, if the level of the lung cancer risk score is between the first threshold and the second threshold levels.

In some embodiments, the first diagnostic intervention comprises performing a transthoracic needle aspiration, mediastinoscopy or thoracotomy. In some embodiments, the second diagnostic intervention comprises engaging in watchful waiting (e.g., periodic monitoring). In some embodiments, watchful waiting comprises periodically imaging the respiratory tract to evaluate the suspicious lesion. In some embodiments, watchful waiting comprises periodically imaging the respiratory tract to evaluate the suspicious lesion for up to one year, two years, four years, five years or more. In some embodiments, watchful waiting comprises imaging the respiratory tract to evaluate the suspicious lesion at least once per year. In some embodiments, watchful waiting comprises imaging the respiratory tract to evaluate the suspicious lesion at least twice per year. In some embodiments, watchful waiting comprises periodic monitoring of a subject unless and until the subject is diagnosed as being free of cancer. In some embodiments, watchful waiting comprises periodic monitoring of a subject unless and until the subject is diagnosed as having cancer. In some embodiments, watchful waiting comprises periodically repeating one or more of steps (a) to (f). In some embodiments, the third diagnostic intervention comprises performing a bronchoscopy procedure. In some embodiments, the third diagnostic intervention comprises repeating steps (a) to (e). In certain embodiments, the third diagnostic intervention comprises repeating steps (a) to (e) within six months of determining that the lung cancer risk score is between the first threshold and the second threshold levels. In certain embodiments, the third diagnostic intervention comprises repeating steps (a) to (e) within three months of determining that the lung cancer risk score is between the first threshold and the second threshold levels. In some embodiments, the third diagnostic intervention comprises repeating steps (a) to (e) within one month of determining that the lung cancer risk score is between the first threshold and the second threshold levels.

In some embodiments, the plurality of informative-genes is selected from the group of genes in Tables 4, 7-8, and 9-11. In some embodiments, the expression levels of a subset of these genes are evaluated and compared to reference expression levels (e.g., for normal patients that do not have cancer). In some embodiments, the subset includes a) genes for which an increase in expression is associated with lung cancer or an increased risk for lung cancer, b) genes for which a decrease in expression is associated with lung cancer or an increased risk for lung cancer, or both. In some embodiments, at least 5%, at least 10%, at least 20%, at least 30%, at least 40%, or about 50% of the genes in a subset have an increased level of expression in association with an increased risk for lung cancer. In some embodiments, at least 5%, at least 10%, at least 20%, at least 30%, at least 40%, or about 50% of the genes in a subset have a decreased level of expression in association with an increased risk for lung cancer. In some embodiments, an expression level is evaluated (e.g., assayed or otherwise interrogated) for each of 10-80 or more genes (e.g., 5-10, 10-20, 20-30, 30-40, 40-50, 50-60, 60-70, 70-80, about 10, about 15, about 25, about 35, about 45, about 55, about 65, about 75, or more genes) selected from the genes in Table 7. In some embodiments, the expression levels of the 80 genes in Table 8 are evaluated. In some embodiments, expression levels are evaluated for a subset of the 80 genes in Table 8 (e.g., 5-10, 10-20, 20-30, 30-40, 40-50, 50-60, 60-70, or 70-79, about 10, about 15, about 25, about 35, about 45, about 55, about 65, about 75, of the genes in Table 8). In some embodiments, the expression level of the 36 informative-genes of Table 9 are evaluated. In some embodiments, expression levels are evaluated for a subset of the genes in Table 9 (e.g., 5-10, 10-20, 20-30, 30-35, about 10, about 15, about 25, about 35 genes from the 36 genes of Table 9). In some embodiments, expression levels for one or more control genes also are evaluated (e.g., 1, 2, 3, 4, or 5 of the control genes). It should be appreciated that an assay can also include other genes, for example reference genes or other gene (regardless of how informative they are). However, if the expression profile for any of the informative-gene subsets described herein is indicative of an increased risk for lung cancer, then an appropriate therapeutic or diagnostic recommendation can be made as described herein.

In some embodiments, the identification of changes in expression level of one or more subsets of genes from Tables 7-9 can be provided to a physician or other health care professional in any suitable format. In some embodiments, these gene expression profiles alone may be sufficient for making a diagnosis, providing a prognosis, or for recommending further diagnosis or a particular treatment. However, in some embodiments the gene expression profiles may assist in the diagnosis, prognosis, and/or treatment of a subject along with other information (e.g., other expression information, and/or other physical or chemical information about the subject, including family history).

In some embodiments, a subject is identified as having a suspicious lesion in the respiratory tract by imaging the respiratory tract. In certain embodiments, imaging the respiratory tract comprises performing computer-aided tomography, magnetic resonance imaging, ultrasonography or a chest X-ray.

Methods are provided, in some embodiments, for obtaining biological samples from patients. Expression levels of informative-genes in these biological samples provide a basis for assessing the likelihood that the patient has lung cancer. Methods are provided for processing biological samples. In some embodiments, the processing methods ensure RNA quality and integrity to enable downstream analysis of informative-genes and ensure quality in the results obtained. Accordingly, various quality control steps (e.g., RNA size analyses) may be employed in these methods. Methods are provided for packaging and storing biological samples. Methods are provided for shipping or transporting biological samples, e.g., to an assay laboratory where the biological sample may be processed and/or where a gene expression analysis may be performed. Methods are provided for performing gene expression analyses on biological samples to determine the expression levels of informative-genes in the samples. Methods are provided for analyzing and interpreting the results of gene expression analyses of informative-genes. Methods are provided for generating reports that summarize the results of gene expression analyses, and for transmitting or sending assay results and/or assay interpretations to a health care provider (e.g., a physician). Furthermore, methods are provided for making treatment decisions based on the gene expression assay results, including making recommendations for further treatment or invasive diagnostic procedures.

In some embodiments, aspects of the invention relate to determining the likelihood that a subject has lung cancer, by subjecting a biological sample obtained from a subject to a gene expression analysis, wherein the gene expression analysis comprises determining expression levels in the biological sample of at least one informative-genes (e.g., at least two genes selected from Table 8 or 9), and using the expression levels to assist in determining the likelihood that the subject has lung cancer.

In some embodiments, the step of determining comprises transforming the expression levels into a lung cancer risk-score that is indicative of the likelihood that the subject has lung cancer. In some embodiments, the lung cancer risk-score is the combination of weighted expression levels. In some embodiments, the lung cancer risk-score is the sum of weighted expression levels. In some embodiments, the expression levels are weighted by their relative contribution to predicting increased likelihood of having lung cancer

In some embodiments, aspects of the invention relate to determining a treatment course for a subject, by subjecting a biological sample obtained from the subject to a gene expression analysis, wherein the gene expression analysis comprises determining the expression levels in the biological sample of at least two informative-genes (e.g., at least two mRNAs selected from Table 8 or 9), and determining a treatment course for the subject based on the expression levels. In some embodiments, the treatment course is determined based on a lung cancer risk-score derived from the expression levels. In some embodiments, the subject is identified as a candidate for a lung cancer therapy based on a lung cancer risk-score that indicates the subject has a relatively high likelihood of having lung cancer. In some embodiments, the subject is identified as a candidate for an invasive lung procedure based on a lung cancer risk-score that indicates the subject has a relatively high likelihood of having lung cancer. In some embodiments, the invasive lung procedure is a transthoracic needle aspiration, mediastinoscopy or thoracotomy. In some embodiments, the subject is identified as not being a candidate for a lung cancer therapy or an invasive lung procedure based on a lung cancer risk-score that indicates the subject has a relatively low likelihood of having lung cancer. In some embodiments, a report summarizing the results of the gene expression analysis is created. In some embodiments, the report indicates the lung cancer risk-score.

In some embodiments, aspects of the invention relate to determining the likelihood that a subject has lung cancer by subjecting a biological sample obtained from a subject to a gene expression analysis, wherein the gene expression analysis comprises determining the expression levels in the biological sample of at least one informative-gene (e.g., at least one informative-mRNA selected from Table 8 or 9), and determining the likelihood that the subject has lung cancer based at least in part on the expression levels.

In some embodiments, aspects of the invention relate to determining the likelihood that a subject has lung cancer, by subjecting a biological sample obtained from the respiratory epithelium of a subject to a gene expression analysis, wherein the gene expression analysis comprises determining the expression level in the biological sample of at least one informative-gene (e.g., at least one informative-mRNA selected from Table 8 or 9), and determining the likelihood that the subject has lung cancer based at least in part on the expression level, wherein the biological sample comprises histologically normal tissue.

In some embodiments, aspects of the invention relate to a computer-implemented method for processing genomic information, by obtaining data representing expression levels in a biological sample of at least two informative-genes (e.g., at least two informative-mRNAs from Table 8), wherein the biological sample was obtained of a subject, and using the expression levels to assist in determining the likelihood that the subject has lung cancer. A computer-implemented method can include inputting data via a user interface, computing (e.g., calculating, comparing, or otherwise analyzing) using a processor, and/or outputting results via a display or other user interface.

In some embodiments, the step of determining comprises calculating a risk-score indicative of the likelihood that the subject has lung cancer. In some embodiments, computing the risk-score involves determining the combination of weighted expression levels, wherein the expression levels are weighted by their relative contribution to predicting increased likelihood of having lung cancer. In some embodiments, a computer-implemented method comprises generating a report that indicates the risk-score. In some embodiments, the report is transmitted to a health care provider of the subject.

It should be appreciated that in any embodiment or aspect described herein, a biological sample can be obtained from the respiratory epithelium of the subject. The respiratory epithelium can be of the mouth, nose, pharynx, trachea, bronchi, bronchioles, or alveoli. However, other sources of respiratory epithelium also can be used. The biological sample can comprise histologically normal tissue. The biological sample can be obtained using bronchial brushings, broncho-alveolar lavage, or a bronchial biopsy. The subject can exhibit one or more symptoms of lung cancer and/or have a lesion that is observable by computer-aided tomography or chest X-ray. In some cases, the subject has not been diagnosed with primary lung cancer prior to being evaluating by methods disclosed herein.

In any of the embodiments or aspects described herein, the expression levels can be determined using a quantitative reverse transcription polymerase chain reaction, a bead-based nucleic acid detection assay or an oligonucleotide array assay or other technique.

In any of the embodiments or aspects described herein, the lung cancer can be a adenocarcinoma, squamous cell carcinoma, small cell cancer or non-small cell cancer. In some embodiments, aspects of the invention relate to a composition consisting essentially of at least one nucleic acid probe, wherein each of the at least one nucleic acid probes specifically hybridizes with an informative-gene (e.g., at least one informative-mRNA selected from Table 8 or 9).

In some embodiments, aspects of the invention relate to a composition comprising up to 5, up to 10, up to 25, up to 50, up to 100, or up to 200 nucleic acid probes, wherein each of the nucleic acid probes specifically hybridizes with an informative-gene (e.g., at least one informative-mRNA selected from any of Tables 7-9).

In some embodiments, nucleic acid probes are conjugated directly or indirectly to a bead. In some embodiments, the bead is a magnetic bead. In some embodiments, the nucleic acid probes are immobilized to a solid support. In some embodiments, the solid support is a glass, plastic or silicon chip.

In some embodiments, aspects of the invention relate to a kit comprising at least one container or package housing any nucleic acid probe composition described herein.

In some embodiments, expression levels are determined using a quantitative reverse transcription polymerase chain reaction.

According to some aspects of the invention, kits are provided that comprise primers for amplifying at least two informative-genes selected from Tables 2-4. In some embodiments, the kits (e.g., gene arrays) comprise at least one primer for amplifying at least 1, at least 2, at least 3, at least 4, at least 5, at least 6, at least 7, at least 8, at least 9, at least 10, or at least 20 informative-genes selected from Tables 2-4. In some embodiments, the kits (e.g., gene arrays) comprise at least one primer for amplifying up to 5, up to 10, up to 25, up to 50, up to 100, or up to 200 informative-genes selected from Tables 2-4. In some embodiments, the kits comprise primers that consist essentially of primers for amplifying each of the informative-genes listed in Table 8 or 9. In some embodiments, the gene arrays comprise primers for amplifying one or more control genes, such as ACTB, GAPDH, YWHAZ, POLR2A, DDX3Y or other control genes. In some embodiments, ACTB, GAPDH, YWHAZ, and POLR2A are used as control genes for normalizing expression levels. In some embodiments, DDX3Y is a semi-identity control because it is a gender specific gene, which is generally more highly expressed in males than females. Thus, DDX3Y can be used in some embodiments to determine whether a sample is from a male or female subject. This information can be used to confirm accuracy of personal information about a subject and exclude samples during data analysis if the information is inconsistent with DDX3Y expression information. For example, if personal information indicates that a subject is female but DDX3Y is highly expressed in a sample (indicating a male subject), the sample can be excluded.

These and other aspects are described in more detail herein and are illustrated by the non-limiting figures and examples.

### BRIEF DESCRIPTION OF DRAWINGS

FIG. 1 depicts the results of a reproducibility assessment. The expression of a panel of endogenous control and biomarker genes were analyzed across a set of 11 duplicate dynamic arrays. The coefficient of variation for all genes analyzed was min=0.019, max-0.062.

FIG. 2 provides scatter plots of expression intensities comparing RT-PCR and microarray expression results (Log₂ RQ vs Log₂ Intensity) for both cancer and no-cancer samples.

FIG. 3 provides a scatter plot comparing gene weights determined from microarray expression information and PCR-based expression information for 49 differential expression genes.

FIG. 4 provides a plot of the levels of different performance metrics for prediction models based on different numbers of features. Training and testing was performed using 217 samples and a full PCR data set.

### DETAILED DESCRIPTION OF CERTAIN EMBODIMENTS OF THE INVENTION

In some embodiments, aspects of the invention relate to genes for which expression levels can be used to determine the likelihood that a subject (e.g., a human subject) has lung cancer. In some embodiments, the expression levels (e.g., mRNA levels) of one or more genes described herein can be determined in airway samples (e.g., epithelial cells or other samples obtained during a bronchoscopy or from an appropriate bronchial lavage samples). In some embodiments, the patterns of increased and/or decreased mRNA expression levels for one or more subsets of informative-genes (e.g., 1-5, 5-10, 10-15, 15-20, 20-25, 25-50, 50-80, or more genes) described herein can be determined and used for diagnostic, prognostic, and/or therapeutic purposes. It should be appreciated that one or more expression patterns described herein can be used alone, or can be helpful along with one or more additional patient-specific indicia or symptoms, to provide personalized diagnostic, prognostic, and/or therapeutic predictions or recommendations for a patient. In some embodiments, sets of informative-genes that distinguish smokers (current or former) with and without lung cancer are provided that are useful for predicting the risk of lung cancer with high accuracy. In some embodiments, the informative-genes are selected from Tables 4, 7-8, and 9-11.

In some embodiments, provided herein are methods for establishing appropriate diagnostic intervention plans and/or treatment plans for subjects and for aiding healthcare providers in establishing appropriate diagnostic intervention plans and/or treatment plans. In some embodiments, methods are provided that involve making a risk assessment based on expression levels of informative-genes in a biological sample obtained from a subject during a routine cell or tissue sampling procedure. In some embodiments, methods are provided that involve establishing lung cancer risk scores based on expression levels of informative-genes. In some embodiments, appropriate diagnostic intervention plans are established based at least in part on the lung cancer risk scores. In some embodiments, methods provided herein assist health care providers with making early and accurate diagnoses. In some embodiments, methods provided herein assist health care providers with establishing appropriate therapeutic interventions early on in patients' clinical evaluations. In some embodiments, methods provided herein involve evaluating biological samples obtained during bronchoscopies procedure. In some embodiments, the methods are beneficial because they enable health care providers to make informative decisions regarding patient diagnosis and/or treatment from otherwise uninformative bronchoscopies. In some embodiments, the risk assessment leads to appropriate surveillance for monitoring low risk lesions. In some embodiments, the risk assessment leads to faster diagnosis, and thus, faster therapy for certain cancers.

Provided herein are methods for determining the likelihood that a subject has lung cancer, such as adenocarcinoma, squamous cell carcinoma, small cell cancer or non-small cell cancer. The methods alone or in combination with other methods provide useful information for health care providers to assist them in making diagnostic and therapeutic decisions for a patient. The methods disclosed herein are often employed in instances where other methods have failed to provide useful information regarding the lung cancer status of a patient. For example, approximately 50% of bronchoscopy procedures result in indeterminate or non-diagnostic information. There are multiple sources of indeterminate results, and may depend on the training and procedures available at different medical centers. However, in certain embodiments, molecular methods in combination with bronchoscopy are expected to improve cancer detection accuracy.

Methods disclosed herein provide alternative or complementary approaches for evaluating cell or tissue samples obtained by bronchoscopy procedures (or other procedures for evaluating respiratory tissue), and increase the likelihood that the procedures will result in useful information for managing the patient's care. The methods disclosed herein are highly sensitive, and produce information regarding the likelihood that a subject has lung cancer from cell or tissue samples (e.g., bronchial brushings of airway epithelial cells), which are often obtained from regions in the airway that are remote from malignant lung tissue. In general, the methods disclosed herein involve subjecting a biological sample obtained from a subject to a gene expression analysis to evaluate gene expression levels. However, in some embodiments, the likelihood that the subject has lung cancer is determined in further part based on the results of a histological examination of the biological sample or by considering other diagnostic indicia such as protein levels, mRNA levels, imaging results, chest X-ray exam results etc.

The term “subject,” as used herein, generally refers to a mammal. Typically the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric. The subject may be a smoker, a former smoker or a non-smoker. The subject may have a personal or family history of cancer. The subject may have a cancer-free personal or family history. The subject may exhibit one or more symptoms of lung cancer or other lung disorder (e.g., emphysema, COPD). For example, the subject may have a new or persistent cough, worsening of an existing chronic cough, blood in the sputum, persistent bronchitis or repeated respiratory infections, chest pain, unexplained weight loss and/or fatigue, or breathing difficulties such as shortness of breath or wheezing. The subject may have a lesion, which may be observable by computer-aided tomography or chest X-ray. The subject may be an individual who has undergone a bronchoscopy or who has been identified as a candidate for bronchoscopy (e.g., because of the presence of a detectable lesion or suspicious imaging result). A subject under the care of a physician or other health care provider may be referred to as a “patient.”

Informative-Genes

The expression levels of certain genes have been identified as providing useful information regarding the lung cancer status of a subject. These genes are referred to herein as “informative-genes.” Informative-genes include protein coding genes and non-protein coding genes. It will be appreciated by the skilled artisan that the expression levels of informative-genes may be determined by evaluating the levels of appropriate gene products (e.g., mRNAs, miRNAs, proteins etc.)

Accordingly, the expression levels of certain mRNAs have been identified as providing useful information regarding the lung cancer status of a subject. These mRNAs are referred to herein as “informative-mRNAs.”

Tables 7-9 provide a listing of informative-genes. Table 7 is a list of 225 informative-genes that are differentially expressed in cancer. Table 8 is a list of 80 informative-genes that are differentially expressed in cancer. Table 9 is a list of 36 informative-genes for predicting cancer status and 5 control genes.

In some embodiments, the informative-genes are selected from the group consisting of: BST1, APT12A, DEFB1, C3, TNFAIP2, SOD2, EPHX3, LST1, HCK, CA12, IRAK2, FMNL1, SERPING1, G0S2, and LCP2. In some embodiments, the informative-genes are selected from the group consisting of: TMTC2, SCHIP1, NMUR2, SORBS2, NPAS2, AKAP12, CSDA, SH3BGRL2, CD9, C9orf102, GRIK2, CAPN9, C19orf2, PRSS23, CA12, NCL, FUT8, PAWR, MTERFD3, RMND5A, OXR1, ALG1L, DAAM1, SLC26A2, AGPS, HDGFRP3, PLCB4, PAM, FOXJ3, TSPAN5, EDEM3, DEFB1, SLC17A5, ZBTB34, MYO1E, MIA3, and ZNF12. In some embodiments, the informative-genes are selected from the group consisting of: EPHX3, HLA-DQB2, BST1, ATP12A, HLA-DQB2, C3, CD82, INSR, PTPN7, FMNL1, IKBKE, RAC2, NINJ1, HLA-DPB1, MDK, ACSS2, HCK, GPRC5B, IRAK2, PLEK, COTL1, CYTH4, TNFAIP2, SCNN1B, LCP2, SOD2, HLA-DMB, CMTM1, SERPING1, CIITA, LILRA5, REC8, CORO1A, LST1, P2RY13, NCF4, G0S2, and TMC6. In some embodiments, the informative-genes are selected from the group consisting of: ACSS2, AKAP12, ATP12A, BST1, C3, CA12, CA8, CCDC81, CD82, EPHX3, ETS1, GPRC5B, HLA-DQB2, INSR, LOC339524, NKX3-1, NMUR2, SH3BGRL2, SLAMF7, and TSPAN5.

Certain methods disclosed herein involve determining expression levels in the biological sample of at least one informative-gene. However, in some embodiments, the expression analysis involves determining the expression levels in the biological sample of at least 2, at least 3, at least 4, at least 5, at least 6, at least 7, at least 8, at least 9, at least 10, at least 20, at least 30, at least 40, at least 50, at least 60, at least 70, or least 80 informative-genes.

In some embodiments, the number of informative-genes for an expression analysis are sufficient to provide a level of confidence in a prediction outcome that is clinically useful. This level of confidence (e.g., strength of a prediction model) may be assessed by a variety of performance parameters including, but not limited to, the accuracy, sensitivity specificity, and area under the curve (AUC) of the receiver operator characteristic (ROC). These parameters may be assessed with varying numbers of features (e.g., number of genes, mRNAs) to determine an optimum number and set of informative-genes. An accuracy, sensitivity or specificity of at least 60%, 70%, 80%, 90%, may be useful when used alone or in combination with other information.

Any appropriate system or method may be used for determining expression levels of informative-genes. Gene expression levels may be determined through the use of a hybridization-based assay. As used herein, the term, “hybridization-based assay” refers to any assay that involves nucleic acid hybridization. A hybridization-based assay may or may not involve amplification of nucleic acids. Hybridization-based assays are well known in the art and include, but are not limited to, array-based assays (e.g., oligonucleotide arrays, microarrays), oligonucleotide conjugated bead assays (e.g., Multiplex Bead-based Luminex® Assays), molecular inversion probe assays, and quantitative RT-PCR assays. Multiplex systems, such as oligonucleotide arrays or bead-based nucleic acid assay systems are particularly useful for evaluating levels of a plurality of genes simultaneously. Other appropriate methods for determining levels of nucleic acids will be apparent to the skilled artisan.

As used herein, a “level” refers to a value indicative of the amount or occurrence of a substance, e.g., an mRNA. A level may be an absolute value, e.g., a quantity of mRNA in a sample, or a relative value, e.g., a quantity of mRNA in a sample relative to the quantity of the mRNA in a reference sample (control sample). The level may also be a binary value indicating the presence or absence of a substance. For example, a substance may be identified as being present in a sample when a measurement of the quantity of the substance in the sample, e.g., a fluorescence measurement from a PCR reaction or microarray, exceeds a background value. Similarly, a substance may be identified as being absent from a sample (or undetectable in the sample) when a measurement of the quantity of the molecule in the sample is at or below background value. It should be appreciated that the level of a substance may be determined directly or indirectly.

Further non-limiting examples of informative mRNAs are disclosed in, for example, the following patent applications, the contents of which are incorporated herein by reference in their entirety for all purposes: U.S. Patent Publication No. US2007/148650, filed on May 12, 2006, entitled ISOLATION OF NUCLEIC ACID FROM MOUTH EPITHELIAL CELLS; U.S. Patent Publication No. US2009/311692, filed Jan. 9, 2009, entitled ISOLATION OF NUCLEIC ACID FROM MOUTH EPITHELIAL CELLS; U.S. application Ser. No. 12/884,714, filed Sep. 17, 2010, entitled ISOLATION OF NUCLEIC ACID FROM MOUTH EPITHELIAL CELLS; U.S. Patent Publication No. US2006/154278, filed Dec. 6, 2005, entitled DETECTION METHODS FOR DISORDER OF THE LUNG; U.S. Patent Publication No. US2010/035244, filed Feb. 8, 2008, entitled, DIAGNOSTIC FOR LUNG DISORDERS USING CLASS PREDICTION; U.S. application Ser. No. 12/869,525, filed Aug. 26, 2010, entitled, DIAGNOSTIC FOR LUNG DISORDERS USING CLASS PREDICTION; U.S. application Ser. No. 12/234,368, filed Sep. 19, 2008, entitled, BIOMARKERS FOR SMOKE EXPOSURE; U.S. application Ser. No. 12/905,897, filed Oct. 154, 2010, entitled BIOMARKERS FOR SMOKE EXPOSURE; U.S. Patent Application No. US2009/186951, filed Sep. 19, 2008, entitled IDENTIFICATION OF NOVEL PATHWAYS FOR DRUG DEVELOPMENT FOR LUNG DISEASE; U.S. Publication No. US2009/061454, filed Sep. 9, 2008, entitled, DIAGNOSTIC AND PROGNOSTIC METHODS FOR LUNG DISORDERS USING GENE EXPRESSION PROFILES; U.S. application Ser. No. 12/940,840, filed Nov. 5, 2010, entitled, DIAGNOSTIC AND PROGNOSTIC METHODS FOR LUNG DISORDERS USING GENE EXPRESSION PROFILES; and U.S. Publication No. US2010/055689, filed Mar. 30, 2009, entitled, MULTIFACTORIAL METHODS FOR DETECTING LUNG DISORDERS.

Biological Samples

The methods generally involve obtaining a biological sample from a subject. As used herein, the phrase “obtaining a biological sample” refers to any process for directly or indirectly acquiring a biological sample from a subject. For example, a biological sample may be obtained (e.g., at a point-of-care facility, a physician's office, a hospital) by procuring a tissue or fluid sample from a subject. Alternatively, a biological sample may be obtained by receiving the sample (e.g., at a laboratory facility) from one or more persons who procured the sample directly from the subject.

The term “biological sample” refers to a sample derived from a subject, e.g., a patient. A biological sample typically comprises a tissue, cells and/or biomolecules. In some embodiments, a biological sample is obtained on the basis that it is histologically normal, e.g., as determined by endoscopy, e.g., bronchoscopy. In some embodiments, biological samples are obtained from a region, e.g., the bronchus or other area or region, that is not suspected of containing cancerous cells. In some embodiments, a histological or cytological examination is performed. However, it should be appreciated that a histological or cytological examination may be optional. In some embodiments, the biological sample is a sample of respiratory epithelium. The respiratory epithelium may be of the mouth, nose, pharynx, trachea, bronchi, bronchioles, or alveoli of the subject. The biological sample may comprise epithelium of the bronchi. In some embodiments, the biological sample is free of detectable cancer cells, e.g., as determined by standard histological or cytological methods. In some embodiments, histologically normal samples are obtained for evaluation. Often biological samples are obtained by scrapings or brushings, e.g., bronchial brushings. However, it should be appreciated that other procedures may be used, including, for example, brushings, scrapings, broncho-alveolar lavage, a bronchial biopsy or a transbronchial needle aspiration.

It is to be understood that a biological sample may be processed in any appropriate manner to facilitate determining expression levels. For example, biochemical, mechanical and/or thermal processing methods may be appropriately used to isolate a biomolecule of interest, e.g., RNA, from a biological sample. Accordingly, a RNA or other molecules may be isolated from a biological sample by processing the sample using methods well known in the art.

Lung Cancer Assessment

Methods disclosed herein may involve comparing expression levels of informative-genes with one or more appropriate references. An “appropriate reference” is an expression level (or range of expression levels) of a particular informative-gene that is indicative of a known lung cancer status. An appropriate reference can be determined experimentally by a practitioner of the methods or can be a pre-existing value or range of values. An appropriate reference represents an expression level (or range of expression levels) indicative of lung cancer. For example, an appropriate reference may be representative of the expression level of an informative-gene in a reference (control) biological sample obtained from a subject who is known to have lung cancer. When an appropriate reference is indicative of lung cancer, a lack of a detectable difference (e.g., lack of a statistically significant difference) between an expression level determined from a subject in need of characterization or diagnosis of lung cancer and the appropriate reference may be indicative of lung cancer in the subject. When an appropriate reference is indicative of lung cancer, a difference between an expression level determined from a subject in need of characterization or diagnosis of lung cancer and the appropriate reference may be indicative of the subject being free of lung cancer.

Alternatively, an appropriate reference may be an expression level (or range of expression levels) of a gene that is indicative of a subject being free of lung cancer. For example, an appropriate reference may be representative of the expression level of a particular informative-gene in a reference (control) biological sample obtained from a subject who is known to be free of lung cancer. When an appropriate reference is indicative of a subject being free of lung cancer, a difference between an expression level determined from a subject in need of diagnosis of lung cancer and the appropriate reference may be indicative of lung cancer in the subject. Alternatively, when an appropriate reference is indicative of the subject being free of lung cancer, a lack of a detectable difference (e.g., lack of a statistically significant difference) between an expression level determined from a subject in need of diagnosis of lung cancer and the appropriate reference level may be indicative of the subject being free of lung cancer.

In some embodiments, the reference standard provides a threshold level of change, such that if the expression level of a gene in a sample is within a threshold level of change (increase or decrease depending on the particular marker) then the subject is identified as free of lung cancer, but if the levels are above the threshold then the subject is identified as being at risk of having lung cancer.

In some embodiments, the methods involve comparing the expression level of an informative-gene to a reference standard that represents the expression level of the informative-gene in a control subject who is identified as not having lung cancer. This reference standard may be, for example, the average expression level of the informative-gene in a population of control subjects who are identified as not having lung cancer.

The magnitude of difference between a expression level and an appropriate reference that is statistically significant may vary. For example, a significant difference that indicates lung cancer may be detected when the expression level of an informative-gene in a biological sample is at least 1%, at least 5%, at least 10%, at least 25%, at least 50%, at least 100%, at least 250%, at least 500%, or at least 1000% higher, or lower, than an appropriate reference of that gene. Similarly, a significant difference may be detected when the expression level of informative-gene in a biological sample is at least 1.1-fold, 1.2-fold, 1.5-fold, 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference of that gene. In some embodiments, at least a 20% to 50% difference in expression between an informative-gene and appropriate reference is significant. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

It is to be understood that a plurality of expression levels may be compared with plurality of appropriate reference levels, e.g., on a gene-by-gene basis, in order to assess the lung cancer status of the subject. The comparison may be made as a vector difference. In such cases, Multivariate Tests, e.g., Hotelling's T² test, may be used to evaluate the significance of observed differences. Such multivariate tests are well known in the art and are exemplified in Applied Multivariate Statistical Analysis by Richard Arnold Johnson and Dean W. Wichern Prentice Hall; 6^{th} edition (Apr. 2, 2007).

Classification Methods

The methods may also involve comparing a set of expression levels (referred to as an expression pattern or profile) of informative-genes in a biological sample obtained from a subject with a plurality of sets of reference levels (referred to as reference patterns), each reference pattern being associated with a known lung cancer status, identifying the reference pattern that most closely resembles the expression pattern, and associating the known lung cancer status of the reference pattern with the expression pattern, thereby classifying (characterizing) the lung cancer status of the subject.

The methods may also involve building or constructing a prediction model, which may also be referred to as a classifier or predictor, that can be used to classify the disease status of a subject. As used herein, a “lung cancer-classifier” is a prediction model that characterizes the lung cancer status of a subject based on expression levels determined in a biological sample obtained from the subject. Typically the model is built using samples for which the classification (lung cancer status) has already been ascertained. Once the model (classifier) is built, it may then be applied to expression levels obtained from a biological sample of a subject whose lung cancer status is unknown in order to predict the lung cancer status of the subject. Thus, the methods may involve applying a lung cancer-classifier to the expression levels, such that the lung cancer-classifier characterizes the lung cancer status of a subject based on the expression levels. The subject may be further treated or evaluated, e.g., by a health care provider, based on the predicted lung cancer status.

The classification methods may involve transforming the expression levels into a lung cancer risk-score that is indicative of the likelihood that the subject has lung cancer. In some embodiments, such as, for example, when a linear discriminant classifier is used, the lung cancer risk-score may be obtained as the combination (e.g., sum, product, or other combination) of weighted expression levels, in which the expression levels are weighted by their relative contribution to predicting increased likelihood of having lung cancer.

It should be appreciated that a variety of prediction models known in the art may be used as a lung cancer-classifier. For example, a lung cancer-classifier may comprises an algorithm selected from logistic regression, partial least squares, linear discriminant analysis, quadratic discriminant analysis, neural network, naïve Bayes, C4.5 decision tree, k-nearest neighbor, random forest, support vector machine, or other appropriate method.

The lung cancer-classifier may be trained on a data set comprising expression levels of the plurality of informative-genes in biological samples obtained from a plurality of subjects identified as having lung cancer. For example, the lung cancer-classifier may be trained on a data set comprising expression levels of a plurality of informative-genes in biological samples obtained from a plurality of subjects identified as having lung cancer based histological findings. The training set will typically also comprise control subjects identified as not having lung cancer. As will be appreciated by the skilled artisan, the population of subjects of the training data set may have a variety of characteristics by design, e.g., the characteristics of the population may depend on the characteristics of the subjects for whom diagnostic methods that use the classifier may be useful. For example, the population may consist of all males, all females or may consist of both males and females. The population may consist of subjects with history of cancer, subjects without a history of cancer, or a subjects from both categories. The population may include subjects who are smokers, former smokers, and/or non-smokers.

A class prediction strength can also be measured to determine the degree of confidence with which the model classifies a biological sample. This degree of confidence may serve as an estimate of the likelihood that the subject is of a particular class predicted by the model. Accordingly, the prediction strength conveys the degree of confidence of the classification of the sample and evaluates when a sample cannot be classified. There may be instances in which a sample is tested, but does not belong, or cannot be reliably assigned to, a particular class. This may be accomplished, for example, by utilizing a threshold, or range, wherein a sample which scores above or below the determined threshold, or within the particular range, is not a sample that can be classified (e.g., a “no call”).

Once a model is built, the validity of the model can be tested using methods known in the art. One way to test the validity of the model is by cross-validation of the dataset. To perform cross-validation, one, or a subset, of the samples is eliminated and the model is built, as described above, without the eliminated sample, forming a “cross-validation model.” The eliminated sample is then classified according to the model, as described herein. This process is done with all the samples, or subsets, of the initial dataset and an error rate is determined. The accuracy the model is then assessed. This model classifies samples to be tested with high accuracy for classes that are known, or classes have been previously ascertained. Another way to validate the model is to apply the model to an independent data set, such as a new biological sample having an unknown lung cancer status.

As will be appreciated by the skilled artisan, the strength of the model may be assessed by a variety of parameters including, but not limited to, the accuracy, sensitivity and specificity. Methods for computing accuracy, sensitivity and specificity are known in the art and described herein (See, e.g., the Examples). The lung cancer-classifier may have an accuracy of at least 60%, at least 65%, at least 70%, at least 75%, at least 80%, at least 85%, at least 90%, at least 95%, at least 99%, or more. The lung cancer-classifier may have an accuracy in a range of about 60% to 70%, 70% to 80%, 80% to 90%, or 90% to 100%. The lung cancer-classifier may have a sensitivity of at least 60%, at least 65%, at least 70%, at least 75%, at least 80%, at least 85%, at least 90%, at least 95%, at least 99%, or more. The lung cancer-classifier may have a sensitivity in a range of about 60% to 70%, 70% to 80%, 80% to 90%, or 90% to 100%. The lung cancer-classifier may have a specificity of at least 60%, at least 65%, at least 70%, at least 75%, at least 80%, at least 85%, at least 90%, at least 95%, at least 99%, or more. The lung cancer-classifier may have a specificity in a range of about 60% to 70%, 70% to 80%, 80% to 90%, or 90% to 100%.

Clinical Treatment/Management

In certain aspects, methods are provided for determining a treatment course for a subject. The methods typically involve determining the expression levels in a biological sample obtained from the subject of one or more informative-genes, and determining a treatment course for the subject based on the expression levels. Often the treatment course is determined based on a lung cancer risk-score derived from the expression levels. The subject may be identified as a candidate for a lung cancer therapy based on a lung cancer risk-score that indicates the subject has a relatively high likelihood of having lung cancer. The subject may be identified as a candidate for an invasive lung procedure (e.g., transthoracic needle aspiration, mediastinoscopy, or thoracotomy) based on a lung cancer risk-score that indicates the subject has a relatively high likelihood of having lung cancer (e.g., greater than 60%, greater than 70%, greater than 80%, greater than 90%). The subject may be identified as not being a candidate for a lung cancer therapy or an invasive lung procedure based on a lung cancer risk-score that indicates the subject has a relatively low likelihood (e.g., less than 50%, less than 40%, less than 30%, less than 20%) of having lung cancer. In some cases, an intermediate risk-score is obtained and the subject is not indicated as being in the high risk or the low risk categories. In some embodiments, a health care provider may engage in “watchful waiting” and repeat the analysis on biological samples taken at one or more later points in time, or undertake further diagnostics procedures to rule out lung cancer, or make a determination that cancer is present, soon after the risk determination was made. The methods may also involve creating a report that summarizes the results of the gene expression analysis. Typically the report would also include an indication of the lung cancer risk-score.

Computer Implemented Methods

Methods disclosed herein may be implemented in any of numerous ways. For example, certain embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the invention may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

As used herein, the term “database” generally refers to a collection of data arranged for ease and speed of search and retrieval. Further, a database typically comprises logical and physical data structures. Those skilled in the art will recognize the methods described herein may be used with any type of database including a relational database, an object-relational database and an XML-based database, where XML stands for “eXtensible-Markup-Language”. For example, the gene expression information may be stored in and retrieved from a database. The gene expression information may be stored in or indexed in a manner that relates the gene expression information with a variety of other relevant information (e.g., information relevant for creating a report or document that aids a physician in establishing treatment protocols and/or making diagnostic determinations, or information that aids in tracking patient samples). Such relevant information may include, for example, patient identification information, ordering physician identification information, information regarding an ordering physician's office (e.g., address, telephone number), information regarding the origin of a biological sample (e.g., tissue type, date of sampling), biological sample processing information, sample quality control information, biological sample storage information, gene annotation information, lung-cancer risk classifier information, lung cancer risk factor information, payment information, order date information, etc.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

In some aspects of the invention, computer implemented methods for processing genomic information are provided. The methods generally involve obtaining data representing expression levels in a biological sample of one or more informative-genes and determining the likelihood that the subject has lung cancer based at least in part on the expression levels. Any of the statistical or classification methods disclosed herein may be incorporated into the computer implemented methods. In some embodiments, the methods involve calculating a risk-score indicative of the likelihood that the subject has lung cancer. Computing the risk-score may involve a determination of the combination (e.g., sum, product or other combination) of weighted expression levels, in which the expression levels are weighted by their relative contribution to predicting increased likelihood of having lung cancer. The computer implemented methods may also involve generating a report that summarizes the results of the gene expression analysis, such as by specifying the risk-score. Such methods may also involve transmitting the report to a health care provider of the subject.

Compositions and Kits

In some aspects, compositions and related methods are provided that are useful for determining expression levels of informative-genes. For example, compositions are provided that consist essentially of nucleic acid probes that specifically hybridize with informative-genes or with nucleic acids having sequences complementary to informative-genes. These compositions may also include probes that specifically hybridize with control genes or nucleic acids complementary thereto. These compositions may also include appropriate buffers, salts or detection reagents. The nucleic acid probes may be fixed directly or indirectly to a solid support (e.g., a glass, plastic or silicon chip) or a bead (e.g., a magnetic bead). The nucleic acid probes may be customized for used in a bead-based nucleic acid detection assay.

In some embodiments, compositions are provided that comprise up to 5, up to 10, up to 25, up to 50, up to 100, or up to 200 nucleic acid probes. In some cases, each of the nucleic acid probes specifically hybridizes with an mRNA selected from Table 7 or with a nucleic acid having a sequence complementary to the mRNA. In some embodiments, probes that detect informative-mRNAs are also included. In some cases, each of at least 2, at least 3, at least 4, at least 5, at least 6, at least 7, at least 8, at least 9, at least 10, or at least 20 of the nucleic acid probes specifically hybridizes with an mRNA selected from Table 8 or 9 or with a nucleic acid having a sequence complementary to the mRNA. In some embodiments, the compositions are prepared for detecting different genes in biochemically separate reactions, or for detecting multiple genes in the same biochemical reactions. In some embodiments, the compositions are prepared for performing a multiplex reaction.

Also provided herein are oligonucleotide (nucleic acid) arrays that are useful in the methods for determining levels of multiple informative-genes simultaneously. Such arrays may be obtained or produced from commercial sources. Methods for producing nucleic acid arrays are also well known in the art. For example, nucleic acid arrays may be constructed by immobilizing to a solid support large numbers of oligonucleotides, polynucleotides, or cDNAs capable of hybridizing to nucleic acids corresponding to genes, or portions thereof. The skilled artisan is referred to Chapter 22 “Nucleic Acid Arrays” of Current Protocols In Molecular Biology (Eds. Ausubel et al. John Wiley and #38; Sons NY, 2000) or Liu CG, et al., An oligonucleotide microchip for genome-wide microRNA profiling in human and mouse tissues. Proc Natl Acad Sci USA. 2004 Jun. 29; 101(26):9740-4, which provide non-limiting examples of methods relating to nucleic acid array construction and use in detection of nucleic acids of interest. In some embodiments, the arrays comprise, or consist essentially of, binding probes for at least 2, at least 5, at least 10, at least 20, at least 50, at least 60, at least 70 or more informative-genes. In some embodiments, the arrays comprise, or consist essentially of, binding probes for up to 2, up to 5, up to 10, up to 20, up to 50, up to 60, up to 70 or more informative-genes. In some embodiments, an array comprises or consists of 1, 2, 3, 4, 5, 6, 7, 8, 9, or 10 of the mRNAs selected from Table 8. In some embodiments, an array comprises or consists of 4, 5, or 6 of the mRNAs selected from Table 8. Kits comprising the oligonucleotide arrays are also provided. Kits may include nucleic acid labeling reagents and instructions for determining expression levels using the arrays.

The compositions described herein can be provided as a kit for determining and evaluating expression levels of informative-genes. The compositions may be assembled into diagnostic or research kits to facilitate their use in diagnostic or research applications. A kit may include one or more containers housing the components of the invention and instructions for use. Specifically, such kits may include one or more compositions described herein, along with instructions describing the intended application and the proper use of these compositions. Kits may contain the components in appropriate concentrations or quantities for running various experiments.

The kit may be designed to facilitate use of the methods described herein by researchers, health care providers, diagnostic laboratories, or other entities and can take many forms. Each of the compositions of the kit, where applicable, may be provided in liquid form (e.g., in solution), or in solid form, (e.g., a dry powder). In certain cases, some of the compositions may be constitutable or otherwise processable, for example, by the addition of a suitable solvent or other substance, which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of diagnostic or biological products, which instructions can also reflect approval by the agency.

A kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The components may be in the form of a liquid, gel or solid (e.g., powder). The components may be prepared sterilely and shipped refrigerated. Alternatively they may be housed in a vial or other container for storage. A second container may have other components prepared sterilely.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

All references described herein are incorporated by reference for the purposes described herein.

Exemplary embodiments of the invention will be described in more detail by the following examples. These embodiments are exemplary of the invention, which one skilled in the art will recognize is not limited to the exemplary embodiments.

### EXAMPLES

### Example 1

### Airway Field of Injury Biomarkers

Introduction:

Applicants have conducted a study to identify airway field of injury biomarkers using RNA recovered from bronchial epithelial cells. Several hundred clinical samples were collected. The samples comprised histologically normal bronchial epithelial cells obtained from the mainstem bronchus during routine bronchoscopy. Subjects from which the samples were obtained were suspected of having lung cancer and were referred to a pulmonologist for bronchoscopy. A subset of the subjects were subsequently confirmed to have lung cancer by histological and pathological examination of cells taken from the lung either during bronchoscopy, or during some follow-up procedure. Another subset of subjects were found to be cancer free at the time of presentation to the pulmonologist and up to 12 months following that date.

The diagnosis of cancer, in all cases, was made by pathology from cells or tissue that were obtained either through bronchoscopy, or in the cases where bronchoscopy was not successful, by follow-up procedures, such as fine-needle aspirate (FNA), surgery (e.g., thoracoscopy, thoracotomy, or mediastinoscopy), or some other technique.

The samples were used to develop a gene expression test to predict subjects with the highest risk of cancer in cases where bronchoscopy yields a non-positive result. The combination of false-negative cases (which occurs in 25-30% of the cancer cases) and the true-negative cases yield a combined set of non-positive bronchoscopy procedures, representing approximately 40-50% of the total cases referred to pulmonologists in this study.

Multivariate analytical strategies, e.g., Linear Discriminant Analysis (LDA) and Support Vector Machine (SVM) were used to generate “scores”. The scores were used to distinguish cancer-positive-positive and cancer-negative cases relative to a threshold. It was found that gene signatures consisting of different numbers of individual genes can lead to effective predictions of cancer. For a given combination of genes the sensitivity and specificity of the algorithm (or signature) was determined by comparison to previously diagnosed cases, with and without cancer. The sensitivity and specificity depends on the threshold value, and a Receiver Operator Characteristic (ROC) curve was constructed.

### Airway Field of Injury Biomarkers

Experiments to evaluate genes associated with airway field of injury have been conducted using gene expression microarrays. A training and testing study was conducted in using a total sample set of 330 clinical specimens. The development set consisted of 240 cancer patients and 90 normal patients (no-cancers). The training set consisted of 220 samples and the independent test set was comprised of 110 samples. Each set consisted of samples from cancers and normal patients. The objective of the training/testing exercise was to determine a useful set of genes (as determined by the probe sets on the array) to predict cancer status. A set of 80 genes (40 up-regulated, and 40 down-regulated) was obtained. These genes were then designated as the candidate gene list for developing and testing Taqman PCR assays.

Taqman assays were selected and first analytically verified by demonstrating which assays had sufficient efficiency and dynamic range. It was found that approximately 90% of the selected assays could be technically verified. Each of the verified assays was then analyzed across a large cohort of clinical specimens (cancers and normal patients) to verify which genes yield optimal clinical sensitivity and specificity. The cohort was chosen as a subset of the 330 samples (described above) that had sufficient RNA remaining.

An objective was to generate PCR data to be used to train and test BronchoGen, similar to what has been done previously using microarray data.

### Summary of Results

Experimental Design—

A total of 229 clinical samples were analyzed using a total of 77 Taqman assays using a Fluidigm Biomark system and dynamic arrays. Each dynamic array is designed with 48 sample wells and 48 assay wells, allowing for a total of 2304 reactions per array. Each assay was analyzed in duplicate, and each array contained control genes in the assay dimension, and control samples in the sample dimension. The total study consisted of approximately 50,000 Taqman assays using 22 dynamic arrays. The breakdown of genes analyzed on each sample is shown in Table 1. Of 229 original samples, a total of 217 samples were analyzed.

TABLE 1

| | |
|---|---|
| Adx Gene | 66 |
| NM gene | 5 |
| HK gene | 4 |
| Gender gene | 2 |
| Final set | 217 |
| Cancers | 152 |
| Normals | 65 |

Table 1 provides experimental design information. RT-PCR was performed using a subset of samples from development set (N=229). A total of ˜50,000 reactions were performed. Fluidigm Biomark system with 48v48 dynamic arrays, requires pre-amplification. 22 arrays were used. Endogenous control genes were present on each array and all reactions were run in duplicate.

Reproducibility:

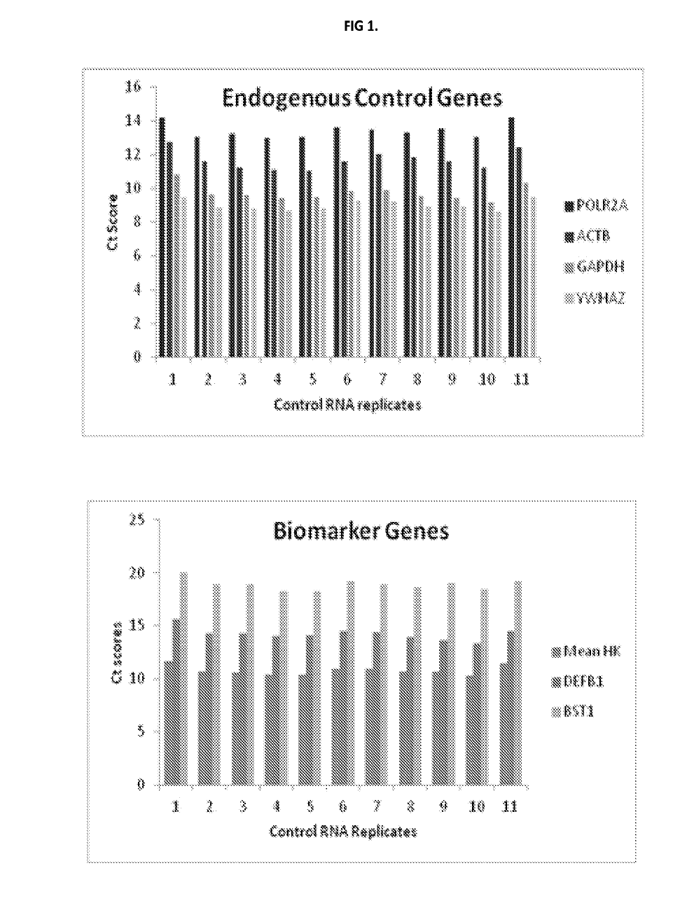

Each sample was analyzed using 77 Taqman assays. Since only 48 assays could be performed on each dynamic array, two arrays were used per set of samples. One of the samples performed on every set of duplicate arrays was a control RNA (prepared by pooling 16 clinical specimens). The reproducibility of the Taqman assays could be assessed by analyzing the 11 replicates of the control RNA. Results are shown in FIG. 1.

Correlation of Expression Intensity:

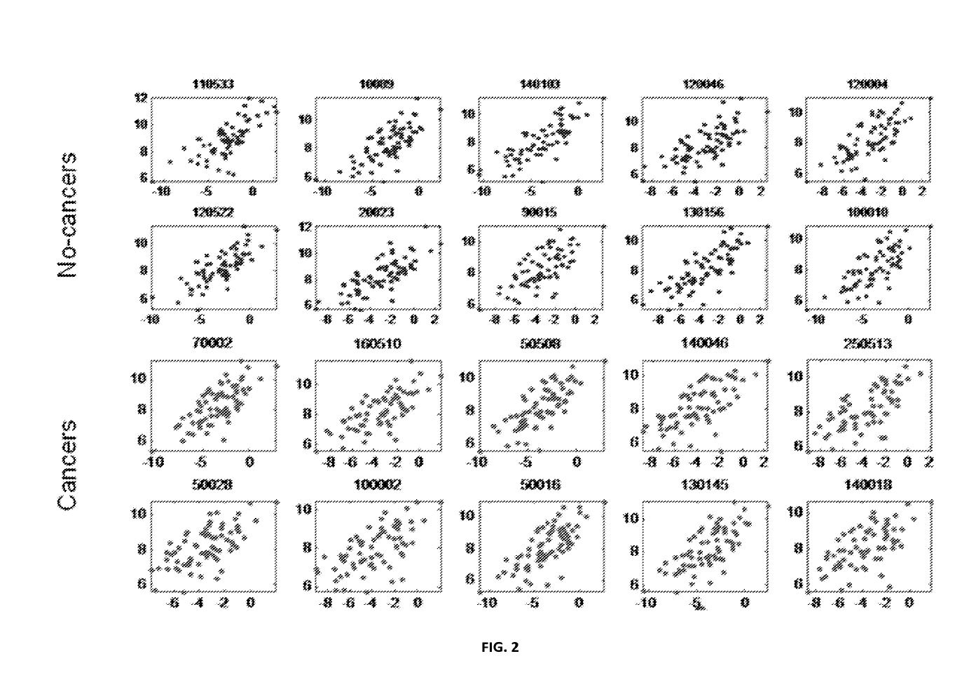

Raw signal intensity from microarray experiments was compared with that from the PCR experiments for the same sample in order to assess the extent of correlation for each of the biomarker candidate genes between the two experimental methods. The plots in FIG. 2 compare the two methods, using Log 2 intensity scales for both detection methods. A collection of 10 randomly chosen cancer and no-cancer samples were selected for the plot in FIG. 2. Good overall correlation is present, which varies somewhat from sample to sample for the individual genes. The range of signal intensities are about twice as large using PCR compared to microarray. The observed correlation was independent of class label (e.g., cancer or no-cancer).

Gene Weights:

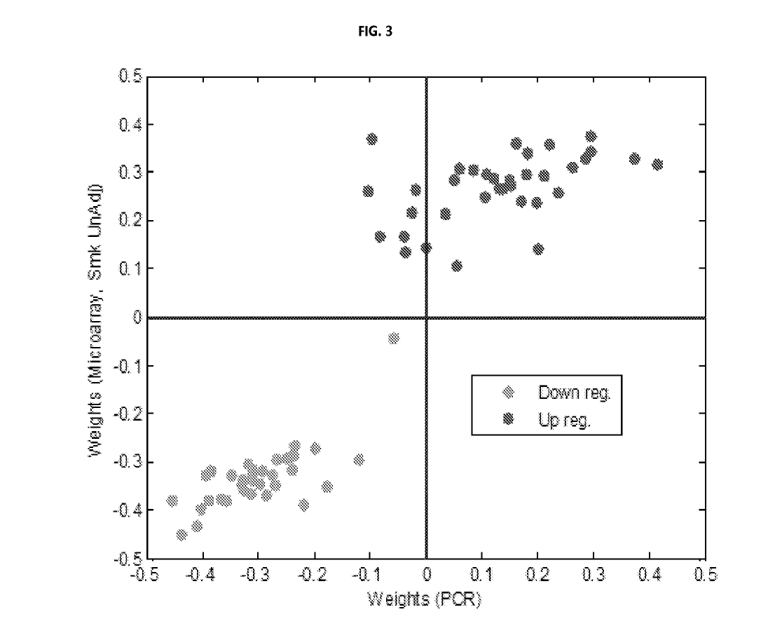

The weight assigned to each gene was determined by calculating the difference in average signal intensity between all cancers and all no-cancers, normalized to the sum of the standard deviation of signal intensity within each class. Weights, therefore provided a “signal to noise” parameter for cancer detection, such that a high positive weight correlated with a high association with cancer status and a high negative weight correlated with a high association with no-cancer status. Each of the candidate genes was selected as having relatively high weights (positive and negative) from the microarray data for the 330 development set. The correlation scatter plot showed very good correlation between microarray and PCR, as shown in FIG. 3. Furthermore, using the PCR data (for the 218 samples), it was found that a total of 49 (of the original 71 biomarker genes) were significantly differentially expressed (p<0.05).

BronchoGen Training/Testing and Prediction Accuracy:

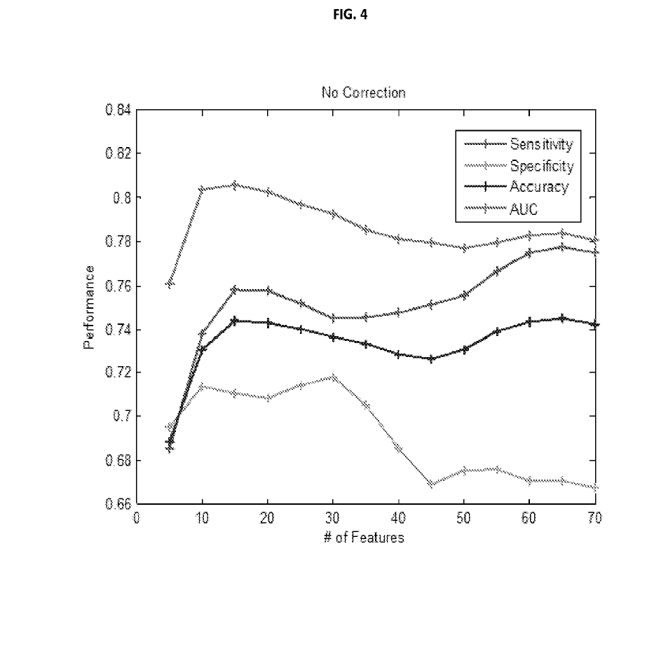

Raw Ct scores for each Taqman assay were converted to relative quantitation (RQ) scores using the standard ΔCt method, and the 4 normalization genes (endogenous controls) run with the dynamic arrays. Analyses of differential expression, and training of an algorithm, were based on the RQ scores. Training and testing of the algorithm was based on an iterative internal cross-validation approach where the total dataset (217 samples) were randomly assigned to training and test set, and then randomized 500 times. The average performance metrics (e.g., sensitivity, specificity) were reported for the 500 iterations, as shown in Table 2. This exercise was also repeated by restricting the number of genes to 5, 10, 15, 20 (etc.) genes in the algorithm, and it was found that, in one embodiment, optimal performance (based on overall area under the ROC curve (AUC)) was obtained using 15 genes, as depicted in FIG. 4. Performance of the algorithm was comparable to what was found using microarray data for the same sample set.

TABLE 2

| | Microarray* | RT-PCR |
|---|---|---|
| Sensitivity | 78% | 76% |
| Specificity | 73% | 71% |
| Accuracy | 76% | 74% |
| AUC | 82% | 81% |

Combined Test Performance:

It was found that for the 215 samples analyzed by PCR (150 cancers versus 65 no-cancers), Bronchoscopy (BR) had a sensitivity of 78%, including TBNA. It was also found that in this example BronchoGen (BG) was complementary to BR and adds approximately 15 percentage points to sensitivity. It was also found to add about 18 percentage points to NPV. However, since NPV is cancer prevalence-dependent and the sample set was skewed with cancers, the NPV was re-calculated assuming a 50% cancer prevalence (e.g., more consistent with a community care hospital), and the NPV was calculated as 91%.

TABLE 3
150 Cancer vs 65 normals

| | BG | BR | BG + BR |
|---|---|---|---|
| Sen | 77.5% | 78.0% | 92.8% |
| Spe7 | 5.5% | 100.0% | 75.5% |
| PPV | 87.7% | 100.0% | 89.5% |
| NPV | 62.5% | 66.3% | 84.4% |
| Accu | 76.9% | 84.7% | 87.3% |
| AUC | 81.6% | | |

Table 3 depicts combined test—bronchoscopy include TBNA, dataset heavily weighted with cancers and balancing for 50% cancer prevalence leads to 91% NPV.

Gene List:

As described above, a useful test accuracy is achieved using on the order of 15 genes. A non-limiting example of 15 useful genes is shown in Table 8 below. The list may be further narrowed to select a smaller set of genes that could still provide prediction accuracy for cancer. Likewise additional genes could be added to provide an algorithm involving 20, 25, 30, or more genes. The non-limiting example of a top 15 gene-set shown in Table 8 includes both up- and down-regulated genes, although the list is heavily dominated with down-regulated genes.

TABLE 4
15 gene-set

| Gene | Weights |
|---|---|
| BST1 | −0.438 |
| APT12A | −0.408 |
| DEFB1 | 0.392 |
| C3 | −0.389 |
| TNFAIP2 | −0.387 |
| SOD2 | −0.373 |
| EPHX3 | −0.369 |
| LST1 | −0.365 |
| HCK | −0.352 |
| CA12 | 0.349 |
| IRAK2 | −0.326 |
| FMNL1 | −0.322 |
| SERPING1 | −0.316 |
| G0S2 | −0.310 |
| LCP2 | −0.306 |

Table 4 depicts an example of a useful gene-list (e.g., for a BronchoGen analysis).

### Example 3

### Biomarkers of Airway Field of Injury

Approximately 1000 specimens were collected for the development and validation of a diagnostic assay (an example of a BronchoGen assay). The specimens were from a mix of subjects with confirmed primary lung cancer, as well as a control group of subjects without lung cancer. Experiments to discover genes associated with airway field of injury were run using gene expression microarrays. An interim analysis exercise was run whereby the first 330 specimens were selected, and the total samples set was split into a training set and a test set, also based on enrollment date and independent of cancer status. The total development set consisted of 240 cancer patients and 90 normal patients (no-cancers). The training set consisted of 220 samples and the independent test set had 110 samples. Each set included samples from cancer patients and normal subjects (without cancer). The objective of the training/testing exercise was to determine a useful set of genes (as determined by the probe sets on the array) to predict cancer status.

The approach of training and testing an algorithm was similar to what had been described previously (Spira, et al., Nature Medicine, 2007). A model was established and the performance was recorded in the training set samples. The algorithm was then locked and used to evaluate the test set. Results of both are shown below in Table 5 based on a total of 80 genes, selected from the top 40 up-regulated and top 40 down-regulated genes in the training set.

TABLE 5

| | Training set | 95% CI | Test set | 95% CI |
|---|---|---|---|---|
| Sen | 79.2% | 72-85% | 73.0% | 63-81% |
| Spe | 70.1% | 58-79% | 76.2% | 55-89% |
| Accu | 76.4% | 70-81% | 73.6% | 65-81% |
| AUC | 81.5% | | 81.4% | |

The training and test samples were then combined to build a model in order to select genes using the most total samples, and therefore maximizing the powering for the gene selection process in this embodiment. The overall prediction accuracy was confirmed to be consistent with the values shown for the training and test sets (above), using a cross-validation approach (Table 6 below). Results are also based on using the top 40 up- and down-regulated genes, in this case based on the combined sample set.

TABLE 6

| | Combined set | 95% CI |
|---|---|---|
| Sen | 78% | 72-83% |
| Spe | 73% | 63-81% |
| Accu | 76% | 71-80% |
| AUC | 81% | |

A t-test was used to determine the total number of differentially expressed genes in the combined sample set (N=330). Using a false-discovery rate (FDR) correction, 796 genes were found to be differentially expressed between cancers (N=240) and non-cancers (N=90), with p<0.05. The majority of differentially expressed genes (N=504; 63%) were down-regulated. A total of 293 (37%) of the differentially expressed genes were up-regulated. In this non-limiting embodiment, in order to build an algorithm using the top 40 up- and top 40 down-regulated genes, the top 225 total differentially expressed genes were evaluated. This list of 225 genes is shown in Table 7. Of these, the top 80 (40 up and 40 down-regulated) are shown in Table 8. The ranking in both tables is based on t-test p-value.

TABLE 7
top 225 total differentially expressed genes

| | | Gene |
|---|---|---|
| Rank | Cluster ID | Symbol |
| 1 | 8034974 | EPHX3 |
| 2 | 8094228 | BST1 |
| 3 | 8180029 | HLA-DQB2 |
| 4 | 7968062 | ATP12A |
| 5 | 8125463 | HLA-DQB2 |
| 6 | 8007757 | FMNL1 |
| 7 | 7957417 | TMTC2 |
| 8 | 8075910 | RAC2 |
| 9 | 7923406 | PTPN7 |
| 10 | 7939546 | CD82 |
| 11 | 8061668 | HCK |
| 12 | 8162455 | NINJ1 |
| 13 | 8179489 | |
| 14 | 8077786 | IRAK2 |
| 15 | 8042391 | PLEK |
| 16 | 8072798 | CYTH4 |
| 17 | 8033257 | C3 |
| 18 | 8062041 | ACSS2 |
| 19 | 7939665 | MDK |
| 20 | 8130556 | SOD2 |
| 21 | 7909188 | IKBKE |
| 22 | 8118594 | HLA-DPB1 |
| 23 | 8104035 | SORBS2 |
| 24 | 8039236 | LILRA5 |
| 25 | 8003171 | COTL1 |
| 26 | 8083677 | SCHIP1 |
| 27 | 8033362 | INSR |
| 28 | 8115734 | LCP2 |
| 29 | 7977046 | TNFAIP2 |
| 30 | 8043909 | NPAS2 |
| 31 | 7909441 | G0S2 |
| 32 | 8091523 | P2RY13 |
| 33 | 8091511 | P2RY14 |
| 34 | 7996290 | CMTM1 |
| 35 | 8072744 | NCF4 |
| 36 | 8179268 | LST1 |
| 37 | 7940028 | SERPING1 |
| 38 | 7994769 | CORO1A |
| 39 | 8156601 | C9orf102 |
| 40 | 7999909 | GPRC5B |
| 41 | 8120833 | SH3BGRL2 |
| 42 | 7910466 | CAPN9 |
| 43 | 8054722 | IL1B |
| 44 | 8036710 | GMFG |
| 45 | 8151512 | PAG1 |
| 46 | 7993195 | CIITA |
| 47 | 8033605 | MYO1F |
| 48 | 8180078 | HLA-DMB |
| 49 | 7961230 | CSDA |
| 50 | 8122807 | AKAP12 |
| 51 | 7995128 | ITGAX |
| 52 | 8121225 | GRIK2 |
| 53 | 8115368 | NMUR2 |
| 54 | 8180022 | |
| 55 | 8125545 | HLA-DOA |
| 56 | 8070826 | ITGB2 |
| 57 | 8088813 | PROK2 |
| 58 | 8034873 | EMR2 |
| 59 | 8027416 | C19orf2 |
| 60 | 8012558 | PIK3R5 |
| 61 | 8075956 | LGALS2 |
| 62 | 7945132 | FLI1 |
| 63 | 8130539 | TAGAP |
| 64 | 7994074 | SCNN1B |
| 65 | 7971461 | LCP1 |
| 66 | 8072757 | CSF2RB |
| 67 | 8000184 | IGSF6 |
| 68 | 7953291 | CD9 |
| 69 | 8145470 | DPYSL2 |
| 70 | 8115490 | ADAM19 |
| 71 | 8035351 | JAK3 |
| 72 | 8036224 | TYROBP |
| 73 | 7906613 | SLAMF7 |
| 74 | 8030277 | CD37 |
| 75 | 7957570 | PLXNC1 |
| 76 | 8147848 | OXR1 |
| 77 | 8104074 | MTNR1A |
| 78 | 7914270 | LAPTM5 |
| 79 | 8018823 | TMC6 |
| 80 | 8003903 | ARRB2 |
| 81 | 7989501 | CA12 |
| 82 | 8036136 | TMEM149 |
| 83 | 8061416 | CST7 |
| 84 | 8169859 | SASH3 |
| 85 | 8063156 | CD40 |
| 86 | 7947861 | SPI1 |
| 87 | 8009653 | CD300A |
| 88 | 7973629 | REC8 |
| 89 | 7921667 | CD48 |
| 90 | 8027862 | FFAR2 |
| 91 | 8179276 | AIF1 |
| 92 | 7926786 | APBB1IP |
| 93 | 7975136 | FUT8 |
| 94 | 8132646 | CCM2 |
| 95 | 7919133 | FCGR1B |
| 96 | 8026971 | IFI30 |
| 97 | 8090291 | ALG1L |
| 98 | 8173444 | IL2RG |
| 99 | 8063497 | CASS4 |
| 100 | 8043310 | RMND5A |
| 101 | 7940869 | FERMT3 |
| 102 | 7942957 | PRSS23 |
| 103 | 8036207 | NFKBID |
| 104 | 8060897 | PLCB4 |
| 105 | 8056860 | WIPF1 |
| 106 | 7971486 | C13orf18 |
| 107 | 7898693 | ALPL |
| 108 | 7902104 | PDE4B |
| 109 | 7974697 | DAAM1 |
| 110 | 7953723 | CLEC4A |
| 111 | 7975889 | VASH1 |
| 112 | 7912937 | PADI2 |
| 113 | 7966046 | MTERFD3 |
| 114 | 8118607 | HLA-DPB2 |
| 115 | 7981530 | GPR132 |
| 116 | 8000482 | XPO6 |
| 117 | 8178295 | UBD |
| 118 | 7906486 | SLAMF8 |
| 119 | 7929911 | LZTS2 |
| 120 | 8179481 | HLA-DRA |
| 121 | 7897877 | TNFRSF1B |
| 122 | 8093624 | SH3BP2 |
| 123 | 7965112 | PAWR |
| 124 | 7952601 | ETS1 |
| 125 | 7927425 | WDFY4 |
| 126 | 8059689 | NCL |
| 127 | 8042637 | DYSF |
| 128 | 8014369 | CCL3 |
| 129 | 7951385 | CASP5 |
| 130 | 8178193 | HLA-DRA |
| 131 | 8178205 | HLA-DQA2 |
| 132 | 8021623 | SERPINB7 |
| 133 | 8180086 | HLA-DMA |
| 134 | 8031374 | FCAR |
| 135 | 7915408 | FOXJ3 |
| 136 | 7997712 | IRF8 |
| 137 | 7906720 | FCER1G |
| 138 | 7892976 | — |
| 139 | 7983478 | C15orf48 |
| 140 | 8115147 | CD74 |
| 141 | 8046604 | AGPS |
| 142 | 7991070 | HDGFRP3 |
| 143 | 8045539 | KYNU |
| 144 | 8031223 | LILRB1 |
| 145 | 8086600 | CCR1 |
| 146 | 8066848 | PREX1 |
| 147 | 7952022 | AMICA1 |
| 148 | 8058905 | IL8RA |
| 149 | 7942439 | RELT |
| 150 | 8107133 | PAM |
| 151 | 7902799 | LOC339524 |
| 152 | 7948332 | LPXN |
| 153 | 7927405 | WDFY4 |
| 154 | 8180356 | — |
| 155 | 8150978 | CA8 |
| 156 | 8075316 | OSM |
| 157 | 8123606 | MGC39372 |
| 158 | 7922823 | EDEM3 |
| 159 | 7990818 | BCL2A1 |
| 160 | 8032410 | MOBKL2A |
| 161 | 7895693 | — |
| 162 | 7963614 | ITGB7 |
| 163 | 7963289 | BIN2 |
| 164 | 8180003 | |
| 165 | 7974341 | GNG2 |
| 166 | 7960865 | SLC2A3 |
| 167 | 8034851 | EMR3 |
| 168 | 8179519 | HLA-DPB1 |
| 169 | 8109194 | SLC26A2 |
| 170 | 8101828 | TSPAN5 |
| 171 | 7903893 | CD53 |
| 172 | 7983490 | C15orf21 |
| 173 | 8138116 | ZNF12 |
| 174 | 8064471 | SIRPB1 |
| 175 | 8157941 | ZBTB34 |
| 176 | 7994826 | ITGAL |
| 177 | 7917576 | GBP5 |
| 178 | 7996318 | CMTM3 |
| 179 | 7893266 | — |
| 180 | 8140319 | HIP1 |
| 181 | 8115783 | STK10 |
| 182 | 8030860 | FPR2 |
| 183 | 7983922 | — |
| 184 | 7899394 | C1orf38 |
| 185 | 8180196 | — |
| 186 | 7905060 | FCGR1A |
| 187 | 8111739 | FYB |
| 188 | 8012013 | CLEC10A |
| 189 | 8073682 | PARVG |
| 190 | 8102594 | TNIP3 |
| 191 | 8016980 | — |
| 192 | 7909371 | CR1 |
| 193 | 8175900 | ARHGAP4 |
| 194 | 8025601 | ICAM1 |
| 195 | 8135436 | SLC26A4 |
| 196 | 8108683 | PCDHB2 |
| 197 | 7989277 | MYO1E |
| 198 | 7909898 | MIA3 |
| 199 | 8018196 | CD300LF |
| 200 | 8127549 | SLC17A5 |
| 201 | 8180411 | — |
| 202 | 8089930 | GOLGB1 |
| 203 | 8156373 | FGD3 |
| 204 | 8053733 | SETD8 |
| 205 | 7958749 | SH2B3 |
| 206 | 8164252 | SH2D3C |
| 207 | 8180263 | — |
| 208 | 7921882 | OLFML2B |
| 209 | 7955908 | NCKAP1L |
| 210 | 7914112 | FGR |
| 211 | 7910398 | RAB4A |
| 212 | 8038899 | FPR1 |
| 213 | 8121515 | SLC16A10 |
| 214 | 7907611 | RASAL2 |
| 215 | 8132819 | IKZF1 |
| 216 | 8094974 | OCIAD1 |
| 217 | 7950906 | CTSC |
| 218 | 8136557 | TBXAS1 |
| 219 | 7996100 | GPR97 |
| 220 | 8123232 | SLC22A1 |
| 221 | 8179041 | |
| 222 | 8109843 | DOCK2 |
| 223 | 8005879 | SLC13A2 |
| 224 | 8056408 | GALNT3 |
| 225 | 8149097 | DEFB1 |

TABLE 8
80 differentially expressed genes

| Top 40 up | | | Top 40 down | | |
|---|---|---|---|---|---|
| Rank | Cluster ID | Gene | Rank | Cluster ID | Gene |
| 7 | 7957417 | TMTC2 | 1 | 8034974 | EPHX3 |
| 26 | 8083677 | SCHIP1 | 3 | 8180029 | HLA-DQB2 |
| 53 | 8115368 | NMUR2 | 2 | 8094228 | BST1 |
| 23 | 8104035 | SORBS2 | 4 | 7968062 | ATP12A |
| 30 | 8043909 | NPAS2 | 5 | 8125463 | HLA-DQB2 |
| 50 | 8122807 | AKAP12 | 17 | 8033257 | C3 |
| 49 | 7961230 | CSDA | 10 | 7939546 | CD82 |
| 41 | 8120833 | SH3BGRL2 | 13 | 8179489 | |
| 68 | 7953291 | CD9 | 27 | 8033362 | INSR |
| 39 | 8156601 | C9orf102 | 9 | 7923406 | PTPN7 |
| 52 | 8121225 | GRIK2 | 6 | 8007757 | FMNL1 |
| 42 | 7910466 | CAPN9 | 21 | 7909188 | IKBKE |
| 59 | 8027416 | C19orf2 | 8 | 8075910 | RAC2 |
| 102 | 7942957 | PRSS23 | 12 | 8162455 | NINJ1 |
| 81 | 7989501 | CA12 | 22 | 8118594 | HLA-DPB1 |
| 126 | 8059689 | NCL | 19 | 7939665 | MDK |
| 93 | 7975136 | FUT8 | 18 | 8062041 | ACSS2 |
| 123 | 7965112 | PAWR | 11 | 8061668 | HCK |
| 113 | 7966046 | MTERFD3 | 40 | 7999909 | GPRC5B |
| 100 | 8043310 | RMND5A | 14 | 8077786 | IRAK2 |
| 76 | 8147848 | OXR1 | 15 | 8042391 | PLEK |
| 97 | 8090291 | ALG1L | 25 | 8003171 | COTL1 |
| 138 | 7892976 | — | 16 | 8072798 | CYTH4 |
| 109 | 7974697 | DAAM1 | 29 | 7977046 | TNFAIP2 |
| 169 | 8109194 | SLC26A2 | 54 | 8180022 | |
| 141 | 8046604 | AGPS | 64 | 7994074 | SCNN1B |
| 142 | 7991070 | HDGFRP3 | 28 | 8115734 | LCP2 |
| 161 | 7895693 | — | 20 | 8130556 | SOD2 |
| 104 | 8060897 | PLCB4 | 48 | 8180078 | HLA-DMB |
| 150 | 8107133 | PAM | 34 | 7996290 | CMTM1 |
| 135 | 7915408 | FOXJ3 | 37 | 7940028 | SERPING1 |
| 170 | 8101828 | TSPAN5 | 46 | 7993195 | CIITA |
| 158 | 7922823 | EDEM3 | 24 | 8039236 | LILRA5 |
| 225 | 8149097 | DEFB1 | 88 | 7973629 | REC8 |
| 200 | 8127549 | SLC17A5 | 38 | 7994769 | CORO1A |
| 175 | 8157941 | ZBTB34 | 36 | 8179268 | LST1 |
| 197 | 7989277 | MYO1E | 32 | 8091523 | P2RY13 |
| 154 | 8180356 | — | 35 | 8072744 | NCF4 |
| 198 | 7909898 | MIA3 | 31 | 7909441 | G0S2 |
| 173 | 8138116 | ZNF12 | 79 | 8018823 | TMC6 |

### Example 2

Custom TaqMan® Low-Density Arrays (TLDAs) have been developed for evaluating informative-genes that are associated airway field of injury. Each custom array comprises a 384-well micro fluidic card. The card permits up to 384 simultaneous real-time PCR reactions. Each card has 8 sample-loading ports, each connected to a set of 48 reaction wells. The reaction protocol involves pipetting a cDNA sample (pre-mixed with an enzyme containing Master Mix) into each sample-loading port and briefly centrifuging. The TLDAs utilize a real-time 5′nuclease fluorescence PCR assay (i.e., TaqMan). In the PCR step, the cDNA templates are amplified using informative-gene specific primers and a fluorescently-labeled hybridization probe.

The informative-genes evaluated in the TLDAs are selected from Table 9. The first 36 genes in Table 9 correspond to informative-genes that differentiate cancers from controls. The last 5 genes, namely ACTB, GAPDH, YWHAZ, POLR2A, and DDX3Y are control genes

In one configuration of the assay, which was used for a validation study, two TLDA cards were used. The first card included primers for each of the genes listed in Table 10 in duplicate within each set of 48 reaction wells, and the second card included primers for each of the genes listed in Table 11 in duplicate within each set of 48 reaction wells. Other configurations of TLDA arrays may be used. For example, other configurations of TLDA arrays that include different combinations of primers for informative-genes may be used.

TABLE 9
Informative-genes for TaqMan ® Low-Density Arrays

| Number | Assay ID | Gene |
|---|---|---|
| 1 | Hs00174709_m1 | BST1 |
| 2 | Hs00196800_m1 | TNFAIP2 |
| 3 | Hs00167309_m1 | SOD2 |
| 4 | Hs00394683_m1 | LST1 |
| 5 | Hs00608345_m1 | DEFB1 |
| 6 | Hs00176654_m1 | HCK |
| 7 | Hs00163811_m1 | C3 |
| 8 | Hs00227184_m1 | EPHX3 |
| 9 | Hs01060284_m1 | ATP12A |
| 10 | Hs01080909_m1 | CA12 |
| 11 | Hs00979762_m1 | FMNL1 |
| 12 | Hs00274783_s1 | G0S2 |
| 13 | Hs00176394_m1 | IRAK2 |
| 14 | Hs00175501_m1 | LCP2 |
| 15 | Hs00163781_m1 | SERPING1 |
| 16 | Hs00173930_m1 | NMUR2 |
| 17 | Hs00374507_m1 | AKAP12 |
| 18 | Hs00974395_m1 | ANXA3 |
| 19 | Hs00220503_m1 | CASS4 |
| 20 | Hs00175188_m1 | CTSC |
| 21 | Hs00265851_m1 | DPYSL2 |
| 22 | Hs00247108_m1 | PADI2 |
| 23 | Hs00171834_m1 | NKX3-1 |
| 24 | Hs01061935_m1 | CACNG4 |
| 25 | Hs00164423_m1 | SLC26A2 |
| 26 | Hs00181751_m1 | GFRA3 |
| 27 | Hs00541345_m1 | TMTC2 |
| 28 | Hs00699550_m1 | TMPRSS11A |
| 29 | Hs00194833_m1 | TSPAN5 |
| 30 | Hs00751478_s1 | S100A10 |
| 31 | Hs00419054_m1 | WDR72 |
| 32 | Hs00322391_m1 | SYNM |
| 33 | Hs00275547_m1 | FCGR3A |
| 34 | Hs00428293_m1 | ETS1 |
| 35 | Hs00172094_m1 | CIITA |
| 36 | Hs01564226_m1 | CCDC81 |
| Controls | | |
| 37 | Hs99999903_m1 | ACTB |
| 38 | Hs02758991_g1 | GAPDH |
| 39 | Hs03044281_g1 | YWHAZ |
| 40 | Hs00172187_m1 | POLR2A |
| 41 | Hs00190539_m1 | DDX3Y |

TABLE 10
TLDA Card 1

| Number | Assay ID | Gene |
|---|---|---|
| 1 | Hs00174709_m1 | BST1 |
| 2 | Hs00196800_m1 | TNFAIP2 |
| 3 | Hs00167309_m1 | SOD2 |
| 4 | Hs00394683_m1 | LST1 |
| 5 | Hs00608345_m1 | DEFB1 |
| 6 | Hs00176654_m1 | HCK |
| 7 | Hs00163811_m1 | C3 |
| 8 | Hs00227184_m1 | EPHX3 |
| 9 | Hs01060284_m1 | ATP12A |
| 10 | Hs01080909_m1 | CA12 |
| 11 | Hs00979762_m1 | FMNL1 |
| 12 | Hs00274783_s1 | G0S2 |
| 13 | Hs00176394_m1 | IRAK2 |
| 14 | Hs00175501_m1 | LCP2 |
| 15 | Hs00163781_m1 | SERPING1 |
| 16 | Hs00173930_m1 | NMUR2 |
| 17 | Hs00374507_m1 | AKAP12 |
| 18 | Hs00974395_m1 | ANXA3 |
| Controls | | |
| 19 | Hs99999903_m1 | ACTB |
| 20 | Hs02758991_g1 | GAPDH |
| 21 | Hs03044281_g1 | YWHAZ |
| 22 | Hs00172187_m1 | POLR2A |
| 23 | Hs00190539_m1 | DDX3Y |

TABLE 11
TLDA Card 2

| Number | Assay ID | Gene |
|---|---|---|
| 1 | Hs00220503_m1 | CASS4 |
| 2 | Hs00175188_m1 | CTSC |
| 3 | Hs00265851_m1 | DPYSL2 |
| 4 | Hs00247108_m1 | PADI2 |
| 5 | Hs00171834_m1 | NKX3-1 |
| 6 | Hs01061935_m1 | CACNG4 |
| 7 | Hs00164423_m1 | SLC26A2 |
| 8 | Hs00181751_m1 | GFRA3 |
| 9 | Hs00541345_m1 | TMTC2 |
| 10 | Hs00699550_m1 | TMPRSS11A |
| 11 | Hs00194833_m1 | TSPAN5 |
| 12 | Hs00751478_s1 | S100A10 |
| 13 | Hs00419054_m1 | WDR72 |
| 14 | Hs00322391_m1 | SYNM |
| 15 | Hs00275547_m1 | FCGR3A |
| 16 | Hs00428293_m1 | ETS1 |
| 17 | Hs00172094_m1 | CIITA |
| 18 | Hs01564226_m1 | CCDC81 |
| Controls | | |
| 19 | Hs99999903_m1 | ACTB |
| 20 | Hs02758991_g1 | GAPDH |
| 21 | Hs03044281_g1 | YWHAZ |
| 22 | Hs00172187_m1 | POLR2A |
| 23 | Hs00190539_m1 | DDX3Y |

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of determining the likelihood that a subject has lung cancer, the method comprising:
subjecting a biological sample obtained from a subject to a gene expression analysis, wherein the gene expression analysis comprises determining mRNA expression levels in the biological sample of at least 1-10 genes selected from Tables 4, 7-8, and 9-11; and
determining the likelihood that the subject has lung cancer by determining a statistical significance on the mRNA expression levels.

2. The method of claim 1, wherein the step of determining the statistical significance comprises transforming the expression levels into a lung cancer risk-score that is indicative of the likelihood that the subject has lung cancer.

3. The method of claim 2, wherein the lung cancer risk-score is the combination of weighted expression levels.

4. The method of claim 3, wherein the lung cancer risk-score is the sum of weighted expression levels.

5. The method of claim 3 or 4, wherein the expression levels are weighted by their relative contribution to predicting increased likelihood of having lung cancer

6. A method for determining a treatment course for a subject, the method comprising:
subjecting a biological sample obtained from the subject to a gene expression analysis, wherein the gene expression analysis comprises determining mRNA expression levels in the biological sample of at least 1-10 genes selected from Tables 4, 7-8, and 9-11;
determining a treatment course for the subject based on the expression levels.

7. The method of claim 6, wherein the treatment course is determined based on a lung cancer risk-score derived from the expression levels.

8. The method of claim 7, wherein the subject is identified as a candidate for a lung cancer therapy based on a lung cancer risk-score that indicates the subject has a relatively high likelihood of having lung cancer.

9. The method of claim 7, wherein the subject is identified as a candidate for an invasive lung procedure based on a lung cancer risk-score that indicates the subject has a relatively high likelihood of having lung cancer.

10. The method of claim 9, wherein the invasive lung procedure is a transthoracic needle aspiration, mediastinoscopy or thoracotomy.

11. The method of claim 7, wherein the subject is identified as not being a candidate for a lung cancer therapy or an invasive lung procedure based on a lung cancer risk-score that indicates the subject has a relatively low likelihood of having lung cancer.

12. The method of any preceding claim further comprising creating a report summarizing the results of the gene expression analysis.

13. The method of any one of claims 2, 3, 7-9, and 11 further comprising creating a report that indicates the lung cancer risk-score.

14. The method of any preceding claim, wherein the biological sample is obtained from the respiratory epithelium of the subject.

15. The method of claim 14, wherein the respiratory epithelium is of the mouth, nose, pharynx, trachea, bronchi, bronchioles, or alveoli.

16. The method of any preceding claim, wherein the biological sample is obtained using bronchial brushings, broncho-alveolar lavage, or a bronchial biopsy.

17. The method of any preceding claim, wherein the subject exhibits one or more symptoms of lung cancer and/or has a lesion that is observable by computer-aided tomography or chest X-ray.

18. The method of claim 17, wherein, prior to subjecting the biological sample to the gene expression analysis, the subject has not be diagnosed with primary lung cancer.

19. The method of any preceding claim, wherein the genes are selected from the group consisting of: BST1, APT12A, DEFB1, C3, TNFAIP2, SOD2, EPHX3, LST1, HCK, CA12, IRAK2, FMNL1, SERPING1, G0S2, and LCP2.

20. The method of any preceding claim, wherein the genes are selected from the group consisting of: TMTC2, SCHIP1, NMUR2, SORBS2, NPAS2, AKAP12, CSDA, SH3BGRL2, CD9, C9orf102, GRIK2, CAPN9, C19orf2, PRSS23, CA12, NCL, FUT8, PAWR, MTERFD3, RMND5A, OXR1, ALG1L, DAAM1, SLC26A2, AGPS, HDGFRP3, PLCB4, PAM, FOXJ3, TSPAN5, EDEM3, DEFB1, SLC17A5, ZBTB34, MYO1E, MIA3, and ZNF12.

21. The method of any preceding claim, wherein the genes are selected from the group consisting of: EPHX3, HLA-DQB2, BST1, ATP12A, HLA-DQB2, C3, CD82, INSR, PTPN7, FMNL1, IKBKE, RAC2, NINJ1, HLA-DPB1, MDK, ACSS2, HCK, GPRC5B, IRAK2, PLEK, COTL1, CYTH4, TNFAIP2, SCNN1B, LCP2, SOD2, HLA-DMB, CMTM1, SERPING1, CIITA, LILRA5, REC8, CORO1A, LST1, P2RY13, NCF4, G0S2, and TMC6.

22. The method of any one of claims 1 to 18, wherein the gene expression analysis comprises determining the expression levels of at least 10 mRNAs expressed from genes selected from Tables 4, 7-8, and 9-11.

23. The method of any one of claims 1 to 18, wherein the gene expression analysis comprises determining the expression levels of at least 15 mRNAs expressed from genes selected from Tables 4, 7-8, and 9-11.

24. The method of any preceding claim wherein the expression levels are determined using a quantitative reverse transcription polymerase chain reaction, a bead-based nucleic acid detection assay or a oligonucleotide array assay.

25. A method of determining the likelihood that a subject has lung cancer, the method comprising:
subjecting a biological sample obtained from a subject to a gene expression analysis, wherein the gene expression analysis comprises determining an mRNA expression level in the biological sample of at least 1 to 10 genes selected from Tables 4, 7-8, and 9-11; and
determining the likelihood that the subject has lung cancer based at least in part on the expression levels.

26. A method of determining the likelihood that a subject has lung cancer, the method comprising:
subjecting a biological sample obtained from the respiratory epithelium of a subject to a gene expression analysis, wherein the gene expression analysis comprises determining an mRNA expression level in the biological sample of at least 1-10 genes selected from Tables 4, 7-8, and 9-11; and
determining the likelihood that the subject has lung cancer based at least in part on the expression level.

27. The method of any preceding claim, wherein the lung cancer is a adenocarcinoma, squamous cell carcinoma, small cell cancer or non-small cell cancer.

28. The method of any preceding claim, wherein the expression level of each of the 15 genes in Table 4 is determined.

29. The method of any preceding claim, wherein the expression levels of at least 2 genes are evaluated.

30. The method of any preceding claim, wherein the expression levels of at least 3 genes are evaluated.

31. The method of any preceding claim, wherein the expression levels of at least 4 genes are evaluated.

32. The method of any preceding claim, wherein the expression levels of at least 5 genes are evaluated.

33. A computer implemented method for processing genomic information, the method comprising:
obtaining data representing expression levels in a biological sample of at least 1-10 mRNAs selected from Tables 4, 7-8, and 9-11, wherein the biological sample was obtained of a subject; and
using the expression levels to assist in determining the likelihood that the subject has lung cancer.

34. The computer implemented method of claim 33, wherein the step of determining comprises calculating a risk-score indicative of the likelihood that the subject has lung cancer.

35. The computer implemented method of claim 34, wherein computing the risk-score involves determining the combination of weighted expression levels, wherein the expression levels are weighted by their relative contribution to predicting increased likelihood of having lung cancer.

36. The computer implemented method of claim 33 furthering comprising generating a report that indicates the risk-score.

37. The computer implemented method of claim 36 further comprising transmitting the report to a health care provider of the subject.

38. The computer implemented method of any one claims 33 to 37, wherein the at least 1-10 mRNAs are selected from the group consisting of: BST1, APT12A, DEFB1, C3, TNFAIP2, SOD2, EPHX3, LST1, HCK, CA12, IRAK2, FMNL1, SERPING1, G0S2, and LCP2.

39. The computer implemented method of any one of claims 33 to 37, wherein the at least 1-10 mRNAs are selected from the group consisting of: TMTC2, SCHIP1, NMUR2, SORBS2, NPAS2, AKAP12, CSDA, SH3BGRL2, CD9, C9orf102, GRIK2, CAPN9, C19orf2, PRSS23, CA12, NCL, FUT8, PAWR, MTERFD3, RMND5A, OXR1, ALG1L, DAAM1, SLC26A2, AGPS, HDGFRP3, PLCB4, PAM, FOXJ3, TSPAN5, EDEM3, DEFB1, SLC17A5, ZBTB34, MYO1E, MIA3, and ZNF12.

40. The computer implemented method of any one of claims 33 to 37, wherein the at least 1-10 mRNAs are selected from the group consisting of: EPHX3, HLA-DQB2, BST1, ATP12A, HLA-DQB2, C3, CD82, INSR, PTPN7, FMNL1, IKBKE, RAC2, NINJ1, HLA-DPB1, MDK, ACSS2, HCK, GPRC5B, IRAK2, PLEK, COTL1, CYTH4, TNFAIP2, SCNN1B, LCP2, SOD2, HLA-DMB, CMTM1, SERPING1, CIITA, LILRA5, REC8, CORO1A, LST1, P2RY13, NCF4, G0S2, and TMC6.

41. The computer implemented method of any one of claims 33 to 37, wherein the gene expression analysis comprises determining mRNA expression levels in an RNA sample of at least 10 genes selected from Tables 4, 7-8, and 9-11.

42. The computer implemented method of any one of claims 33 to 37, wherein the gene expression analysis comprises determining mRNA expression levels in an RNA sample of at least 15 genes selected from Tables 4, 7-8, and 9-11.

43. The computer implemented method of any preceding claim 33-42, wherein the biological sample was obtained from the respiratory epithelium of the subject.

44. A composition consisting essentially of at least 1-10 nucleic acid probes, wherein each of the at least 1-10 nucleic acids probes specifically hybridizes with an mRNA expressed from a different gene selected from the genes of Tables 4, 7-8, and 9-11.

45. A composition comprising up to 5, up to 10, up to 25, up to 50, up to 100, or up to 200 nucleic acid probes, wherein each of at least 1-10 of the nucleic acid probes specifically hybridizes with an mRNA expressed from a different gene selected from the genes of Tables 4, 7-8, and 9-11.

46. The composition of claim 44 or 45, wherein the genes are selected from the group consisting of: BST1, APT12A, DEFB1, C3, TNFAIP2, SOD2, EPHX3, LST1, HCK, CA12, IRAK2, FMNL1, SERPING1, G0S2, and LCP2.

47. The composition of any one of claims 44 to 46, wherein the genes are selected from the group consisting of:
TMTC2, SCHIP1, NMUR2, SORBS2, NPAS2, AKAP12, CSDA, SH3BGRL2, CD9, C9orf102, GRIK2, CAPN9, C19orf2, PRSS23, CA12, NCL, FUT8, PAWR, MTERFD3, RMND5A, OXR1, ALG1L, DAAM1, SLC26A2, AGPS, HDGFRP3, PLCB4, PAM, FOXJ3, TSPAN5, EDEM3, DEFB1, SLC17A5, ZBTB34, MYO1E, MIA3, and ZNF12.

48. The composition of any one of claims 44 to 46, wherein the genes are selected from the group consisting of: EPHX3, HLA-DQB2, BST1, ATP12A, HLA-DQB2, C3, CD82, INSR, PTPN7, FMNL1, IKBKE, RAC2, NINJ1, HLA-DPB1, MDK, ACSS2, HCK, GPRC5B, IRAK2, PLEK, COTL1, CYTH4, TNFAIP2, SCNN1B, LCP2, SOD2, HLA-DMB, CMTM1, SERPING1, CIITA, LILRA5, REC8, CORO1A, LST1, P2RY13, NCF4, G0S2, and TMC6.

49. The composition of any one of claims 44 to 46, wherein each of at least 10 of the nucleic acid probes specifically hybridizes with an mRNA expressed from a gene selected from Tables 4, 7-8, and 9-11 or with a nucleic acid having a sequence complementary to the mRNA.

50. The composition of any one of claims 44 to 46, wherein each of at least 15 of the nucleic acid probes specifically hybridizes with an mRNA expressed from a gene selected from Tables 4, 7-8, and 9-11 or with a nucleic acid having a sequence complementary to the mRNA.

51. The composition of any of claims 44 to 50, wherein the nucleic acid probes are conjugated directly or indirectly to a bead.

52. The composition of any of claims 44 to 50, wherein the bead is a magnetic bead.

53. The composition of any of claims 44 to 51, wherein the nucleic acid probes are immobilized to a solid support.

54. The composition of any of claims 44 to 51, wherein the solid support is a glass, plastic or silicon chip.

55. A kit comprising at least one container or package housing the composition of any one of claims 44 to 50.

56. A method of processing an RNA sample, the method comprising
(a) obtaining an RNA sample;
(b) determining the expression level of a first mRNA in the RNA sample; and
(c) determining the expression level of a second mRNA in the RNA sample, wherein the expression level of the first mRNA and the second mRNA are determined in biochemically separate assays, and wherein the first mRNA and second mRNA are expressed from genes selected from Tables 4, 7-8, and 9-11.

57. The method of claim 56 further comprising determining the expression level of at least one other mRNA in the RNA sample, wherein the expression level of the first mRNA, the second mRNA, and the at least one other mRNA are determined in biochemically separate assays, and wherein the at least one other mRNA is expressed from a gene selected from Tables 4, 7-8, and 9-11.

58. The method of claim 56 or 57, wherein the expression levels are determined using a quantitative reverse transcription polymerase chain reaction.
